SEQUENCE LISTING (116 sequences)

SEQ ID NO: 1 - protein, 5 aa, Artificial Sequence
  Acceptor sequence
LPETG

SEQ ID NO: 2 - protein, 5 aa, Artificial Sequence
  Connection position
  VARIANT 3: Any amino acid
LPXTG

SEQ ID NO: 3 - protein, 7 aa, Artificial Sequence
  Connection position
  VARIANT 5: Any amino acid
GSLPXTG

SEQ ID NO: 4 - protein, 8 aa, Artificial Sequence
  Connection position
  VARIANT 5: Any amino acid
GSLPXTGG

SEQ ID NO: 5 - protein, 9 aa, Artificial Sequence
  Connection position
  VARIANT 5: Any amino acid
GSLPXTGGG

SEQ ID NO: 6 - protein, 10 aa, Artificial Sequence
  Connection position
  VARIANT 5: Any amino acid
GSLPXTGGGG

SEQ ID NO: 7 - protein, 11 aa, Artificial Sequence
  Connection postion
  VARIANT 5: Any amino acid
GSLPXTGGGGG

SEQ ID NO: 8 - protein, 12 aa, Artificial Sequence
  Connection position
  VARIANT 5: Any amino acid
GSLPXTGGGGGG

SEQ ID NO: 9 - protein, 9 aa, Artificial Sequence
  Connection position
  VARIANT 7: Any amino acid
GSGSLPXTG

SEQ ID NO: 10 - protein, 10 aa, Artificial Sequence
  Connection position
  VARIANT 7: Any amino acid
GSGSLPXTGG

SEQ ID NO: 11 - protein, 11 aa, Artificial Sequence
  Connection position
  VARIANT 7: Any amino acid
GSGSLPXTGGG

SEQ ID NO: 12 - protein, 12 aa, Artificial Sequence
  Connection position
  VARIANT 7: Any amino acid
GSGSLPXTGGGG

SEQ ID NO: 13 - protein, 13 aa, Artificial Sequence
  Connection position
  VARIANT 7: Any amino acid
GSGSLPXTGGGGG

SEQ ID NO: 14 - protein, 14 aa, Artificial Sequence
  Connection position
  VARIANT 7: Any amino acid
GSGSLPXTGGGGGG

SEQ ID NO: 15 - protein, 11 aa, Artificial Sequence
  Connection position
  VARIANT 9: Any amino acid
GSGSGSLPXTG

SEQ ID NO: 16 - protein, 12 aa, Artificial Sequence
  Connection position
  VARIANT 9: Any amino acid
GSGSGSLPXTGG

SEQ ID NO: 17 - protein, 13 aa, Artificial Sequence
  Connection position
  VARIANT 9: Any amino acid
GSGSGSLPXTGGG

SEQ ID NO: 18 - protein, 14 aa, Artificial Sequence
  Connection position
  VARIANT 9: Any amino acid
GSGSGSLPXTGGGG

SEQ ID NO: 19 - protein, 15 aa, Artificial Sequence
  Connection position
  VARIANT 9: Any amino acid
GSGSGSLPXTGGGGG

SEQ ID NO: 20 - protein, 16 aa, Artificial Sequence
  Connection position
  VARIANT 9: Any amino acid
GSGSGSLPXTGGGGGG

SEQ ID NO: 21 - protein, 13 aa, Artificial Sequence
  Connection position
  VARIANT 11: Any amino acid
GSGSGSGSLPXTG

SEQ ID NO: 22 - protein, 14 aa, Artificial Sequence
  Connection position
  VARIANT 11: Any amino acid
GSGSGSGSLPXTGG

SEQ ID NO: 23 - protein, 15 aa, Artificial Sequence
  Connection position
  VARIANT 11: Any amino acid
GSGSGSGSLPXTGGG

SEQ ID NO: 24 - protein, 16 aa, Artificial Sequence
  Connection position
  VARIANT 11: Any amino acid
GSGSGSGSLPXTGGGG

SEQ ID NO: 25 - protein, 17 aa, Artificial Sequence
  Connection position
  VARIANT 11: Any amino acid
GSGSGSGSLPXTGGGGG

SEQ ID NO: 26 - protein, 18 aa, Artificial Sequence
  Connection position
  VARIANT 11: Any amino acid
GSGSGSGSLPXTGGGGGG

SEQ ID NO: 27 - protein, 15 aa, Artificial Sequence
  Connection position
  VARIANT 13: Any amino acid
GSGSGSGSGSLPXTG

SEQ ID NO: 28 - protein, 16 aa, Artificial Sequence
  Connection position
  VARIANT 13: Any amino acid
GSGSGSGSGSLPXTGG

SEQ ID NO: 29 - protein, 17 aa, Artificial Sequence
  Connection position
  VARIANT 13: Any amino acid
GSGSGSGSGSLPXTGGG

SEQ ID NO: 30 - protein, 18 aa, Artificial Sequence
  Connection position
  VARIANT 13: Any amino acid
GSGSGSGSGSLPXTGGGG

SEQ ID NO: 31 - protein, 19 aa, Artificial Sequence
  Connection position
  VARIANT 13: Any amino acid
GSGSGSGSGSLPXTGGGGG

SEQ ID NO: 32 - protein, 20 aa, Artificial Sequence
  Connection position
  VARIANT 13: Any amino acid
GSGSGSGSGSLPXTGGGGGG

SEQ ID NO: 33 - protein, 17 aa, Artificial Sequence
  Connection position
  VARIANT 15: Any amino acid
GSGSGSGSGSGSLPXTG

SEQ ID NO: 34 - protein, 18 aa, Artificial Sequence
  Connection position
  VARIANT 15: Any amino acid
GSGSGSGSGSGSLPXTGG

SEQ ID NO: 35 - protein, 19 aa, Artificial Sequence
  Connection position
  VARIANT 15: Any amino acid
GSGSGSGSGSGSLPXTGGG

SEQ ID NO: 36 - protein, 20 aa, Artificial Sequence
  Connection position
  VARIANT 15: Any amino acid
GSGSGSGSGSGSLPXTGGGG

SEQ ID NO: 37 - protein, 21 aa, Artificial Sequence
  Connection position
  VARIANT 15: Any amino acid
GSGSGSGSGSGSLPXTGGGGG

SEQ ID NO: 38 - protein, 22 aa, Artificial Sequence
  Connection position
  VARIANT 15: Any amino acid
GSGSGSGSGSGSLPXTGGGGGG

SEQ ID NO: 39 - protein, 4 aa, Artificial Sequence
  Connection position
HHHH

SEQ ID NO: 40 - protein, 5 aa, Artificial Sequence
  Connection position
HHHHH

SEQ ID NO: 41 - protein, 6 aa, Artificial Sequence
  Connection position
HHHHHH

SEQ ID NO: 42 - protein, 7 aa, Artificial Sequence
  Connection position
HHHHHHH

SEQ ID NO: 43 - protein, 8 aa, Artificial Sequence
  Connection position
HHHHHHHH

SEQ ID NO: 44 - protein, 9 aa, Artificial Sequence
  Connection position
HHHHHHHHH

SEQ ID NO: 45 - protein, 10 aa, Artificial Sequence
  Connection position
HHHHHHHHHH

SEQ ID NO: 46 - DNA, 26 bp, Artificial Sequence
  5′-adaptor sequence
agatcggaagagcacacgtctgaact

SEQ ID NO: 47 - DNA, 66 bp, Artificial Sequence
  Barcode sequence 1 (supplemental table 1)
  misc_feature 27..34: n= A, C, G or T
ttccctacacgacgctcttccgatctnnnnnnnntcactgagatcggaagagcacacgtctgaact

SEQ ID NO: 48 - DNA, 66 bp, Artificial Sequence
  Barcode sequence 2 (supplemental table 1)
  misc_feature 27..34: n = A, C, G or T
ttccctacacgacgctcttccgatctnnnnnnnncttgacagatcggaagagcacacgtctgaact

SEQ ID NO: 49 - DNA, 66 bp, Artificial Sequence
  Barcode sequence 3 (supplemental table 1)
  misc_feature 27..34: n = A, C, G or T
ttccctacacgacgctcttccgatctnnnnnnnnacaggtagatcggaagagcacacgtctgaact

SEQ ID NO: 50 - DNA, 66 bp, Artificial Sequence
  Barcode sequence 4 (supplemental table 1)
  misc_feature 27..34: n = A, C, G or T
ttccctacacgacgctcttccgatctnnnnnnnnggtacaagatcggaagagcacacgtctgaact

SEQ ID NO: 51 - DNA, 66 bp, Artificial Sequence
  Barcode sequence 5 (supplemental table 1)
  misc_feature 27..34: n = A, C, G or T
ttccctacacgacgctcttccgatctnnnnnnnngcgaatagatcggaagagcacacgtctgaact

SEQ ID NO: 52 - DNA, 66 bp, Artificial Sequence
  Barcode sequence 6 (supplemental table 1)
  misc_feature 27..34: n = A, C, G or T
ttccctacacgacgctcttccgatctnnnnnnnncgcttaagatcggaagagcacacgtctgaact

SEQ ID NO: 53 - DNA, 58 bp, Artificial Sequence
  Index primer sequence 1 (supplemental table 1)
caagcagaagacggcatacgagatgctaccgtgactggagttcagacgtgtgctcttc

SEQ ID NO: 54 - DNA, 58 bp, Artificial Sequence
  Index primer sequence 2 (supplemental table 1)
caagcagaagacggcatacgagatgctcatgtgactggagttcagacgtgtgctcttc

SEQ ID NO: 55 - DNA, 58 bp, Artificial Sequence
  Index primer sequence 3 (supplemental table 1)
caagcagaagacggcatacgagataggaatgtgactggagttcagacgtgtgctcttc

SEQ ID NO: 56 - DNA, 58 bp, Artificial Sequence
  Index primer sequence 4 (supplemental table 1)
caagcagaagacggcatacgagattagttggtgactggagttcagacgtgtgctcttc

SEQ ID NO: 57 - DNA, 58 bp, Artificial Sequence
  Index primer sequence 5 (supplemental table 1)
caagcagaagacggcatacgagatatcgtggtgactggagttcagacgtgtgctcttc

SEQ ID NO: 58 - DNA, 58 bp, Artificial Sequence
  Index primer sequence 6 (supplemental table 1)
caagcagaagacggcatacgagatcgattagtgactggagttcagacgtgtgctcttc

SEQ ID NO: 59 - DNA, 58 bp, Artificial Sequence
  Index primer sequence 7 (supplemental table 1)
caagcagaagacggcatacgagatgaatgagtgactggagttcagacgtgtgctcttc

SEQ ID NO: 60 - DNA, 58 bp, Artificial Sequence
  Index primer sequence 8 (supplemental table 1)
caagcagaagacggcatacgagattgccgagtgactggagttcagacgtgtgctcttc

SEQ ID NO: 61 - DNA, 68 bp, Artificial Sequence
  Barcode sequence 1 (table 4)
  misc_feature 27..34: n = A, C, G or T
ttccctacacgacgctcttccgatctnnnnnnnntggcctatagatcggaagagcacacgtctgaact

SEQ ID NO: 62 - DNA, 68 bp, Artificial Sequence
  Barcode sequence 2 (table 4)
  misc_feature 27..34: n = A, C, G or T
ttccctacacgacgctcttccgatctnnnnnnnnacagtatcagatcggaagagcacacgtctgaact

SEQ ID NO: 63 - DNA, 68 bp, Artificial Sequence
  Barcode sequence 3 (table 4)
  misc_feature 27..34: n = A, C, G or T
ttccctacacgacgctcttccgatctnnnnnnnngttaggcaagatcggaagagcacacgtctgaact

SEQ ID NO: 64 - DNA, 68 bp, Artificial Sequence
  Barcode sequence 4 (table 4)
  misc_feature 27..34: n = A, C, G or T
ttccctacacgacgctcttccgatctnnnnnnnncactacggagatcggaagagcacacgtctgaact

SEQ ID NO: 65 - DNA, 68 bp, Artificial Sequence
  Barcode sequence 5 (table 4)
  misc_feature 27..34: n = A, C, G or T
ttccctacacgacgctcttccgatctnnnnnnnntggctagaagatcggaagagcacacgtctgaact

SEQ ID NO: 66 - DNA, 68 bp, Artificial Sequence
  Barcode sequence 6 (table 4)
  misc_feature 27..34: n = A, C, G or T
ttccctacacgacgctcttccgatctnnnnnnnnacagcgctagatcggaagagcacacgtctgaact

SEQ ID NO: 67 - DNA, 68 bp, Artificial Sequence
  Barcode sequence 7 (table 4)
  misc_feature 27..34: n = A, C, G or T
ttccctacacgacgctcttccgatctnnnnnnnngttaactgagatcggaagagcacacgtctgaact

SEQ ID NO: 68 - DNA, 68 bp, Artificial Sequence
  Barcode sequence 8 (table 4)
  misc_feature 27..34: n = A, C, G or T
ttccctacacgacgctcttccgatctnnnnnnnncactgtacagatcggaagagcacacgtctgaact

SEQ ID NO: 69 - DNA, 68 bp, Artificial Sequence
  Barcode sequence 9 (table 4)
  misc_feature 27..34: n = A, C, G or T
ttccctacacgacgctcttccgatctnnnnnnnntgcttacgagatcggaagagcacacgtctgaact

SEQ ID NO: 70 - DNA, 68 bp, Artificial Sequence
  Barcode sequence 10 (table 4)
  misc_feature 27..34: n = A, C, G or T
ttccctacacgacgctcttccgatctnnnnnnnnactcagtaagatcggaagagcacacgtctgaact

SEQ ID NO: 71 - DNA, 68 bp, Artificial Sequence
  Barcode sequence 11 (table 4)
  misc_feature 27..34: n = A, C, G or T
ttccctacacgacgctcttccgatctnnnnnnnncagaccgtagatcggaagagcacacgtctgaact

SEQ ID NO: 72 - DNA, 68 bp, Artificial Sequence
  Barcode sequence 12 (table 4)
  misc_feature 27..34: n = A, C, G or T
ttccctacacgacgctcttccgatctnnnnnnnntgcggagaagatcggaagagcacacgtctgaact

SEQ ID NO: 73 - DNA, 68 bp, Artificial Sequence
  Barcode sequence 13 (table 4)
  misc_feature 27..34: n = A, C, G or T
ttccctacacgacgctcttccgatctnnnnnnnnactactcgagatcggaagagcacacgtctgaact

SEQ ID NO: 74 - DNA, 68 bp, Artificial Sequence
  Barcode sequence 14 (table 4)
  misc_feature 27..34: n = A, C, G or T
ttccctacacgacgctcttccgatctnnnnnnnngtatagtcagatcggaagagcacacgtctgaact

SEQ ID NO: 75 - DNA, 68 bp, Artificial Sequence
  Barcode sequence 15 (table 4)
  misc_feature 27..34: n = A, C, G or T
ttccctacacgacgctcttccgatctnnnnnnnncagctcatagatcggaagagcacacgtctgaact

SEQ ID NO: 76 - DNA, 68 bp, Artificial Sequence
  Barcode sequence 16 (table 4)
  misc_feature 27..34: n = A, C, G or T
ttccctacacgacgctcttccgatctnnnnnnnngtaactccagatcggaagagcacacgtctgaact

SEQ ID NO: 77 - DNA, 59 bp, Artificial Sequence
  Index primer sequence 1 (table 4)
caagcagaagacggcatacgagatggactgagtgactggagttcagacgtgtgctcttc

SEQ ID NO: 78 - DNA, 59 bp, Artificial Sequence
  Index primer sequence 2 (table 4)
caagcagaagacggcatacgagaccattacggtgactggagttcagacgtgtgctcttc

SEQ ID NO: 79 - DNA, 59 bp, Artificial Sequence
  Index primer sequence 3 (table 4)
caagcagaagacggcatacgagagatgacacgtgactggagttcagacgtgtgctcttc

SEQ ID NO: 80 - DNA, 59 bp, Artificial Sequence
  Index primer sequence 4 (table 4)
caagcagaagacggcatacgagaatccggttgtgactggagttcagacgtgtgctcttc

SEQ ID NO: 81 - DNA, 59 bp, Artificial Sequence
  Index primer sequence 5 (table 4)
caagcagaagacggcatacgagacaaggtgagtgactggagttcagacgtgtgctcttc

SEQ ID NO: 82 - DNA, 59 bp, Artificial Sequence
  Index primer sequence 6 (table 4)
caagcagaagacggcatacgagagtgcaacggtgactggagttcagacgtgtgctcttc

SEQ ID NO: 83 - DNA, 59 bp, Artificial Sequence
  Index primer sequence 7 (table 4)
caagcagaagacggcatacgagaagcttcacgtgactggagttcagacgtgtgctcttc

SEQ ID NO: 84 - DNA, 59 bp, Artificial Sequence
  Index primer sequence 8 (table 4)
caagcagaagacggcatacgagatctacgttgtgactggagttcagacgtgtgctcttc

SEQ ID NO: 85 - DNA, 59 bp, Artificial Sequence
  Index primer sequence 9 (table 4)
caagcagaagacggcatacgagagacgaatagtgactggagttcagacgtgtgctcttc

SEQ ID NO: 86 - DNA, 59 bp, Artificial Sequence
  Index primer sequence 10 (table 4)
caagcagaagacggcatacgagacctcctaggtgactggagttcagacgtgtgctcttc

SEQ ID NO: 87 - DNA, 59 bp, Artificial Sequence
  Index primer sequence 11 (table 4)
caagcagaagacggcatacgagaagattcgtgtgactggagttcagacgtgtgctcttc

SEQ ID NO: 88 - DNA, 59 bp, Artificial Sequence
  Index primer sequence 12 (table 4)
caagcagaagacggcatacgagattgaggccgtgactggagttcagacgtgtgctcttc

SEQ ID NO: 89 - DNA, 59 bp, Artificial Sequence
  Index primer sequence 13 (table 4)
caagcagaagacggcatacgagacgagtatagtgactggagttcagacgtgtgctcttc

SEQ ID NO: 90 - DNA, 59 bp, Artificial Sequence
  Index primer sequence 14 (table 4)
caagcagaagacggcatacgagagtccgtaggtgactggagttcagacgtgtgctcttc

SEQ ID NO: 91 - DNA, 59 bp, Artificial Sequence
  Index primer sequence 15 (table 4)
caagcagaagacggcatacgagaacgaacgtgtgactggagttcagacgtgtgctcttc

SEQ ID NO: 92 - DNA, 59 bp, Artificial Sequence
  Index primer sequence 16 (table 4)
caagcagaagacggcatacgagatattcgccgtgactggagttcagacgtgtgctcttc

SEQ ID NO: 93 - DNA, 59 bp, Artificial Sequence
  Index primer sequence 17 (table 4)
caagcagaagacggcatacgagatcgaactagtgactggagttcagacgtgtgctcttc

SEQ ID NO: 94 - DNA, 59 bp, Artificial Sequence
  Index primer sequence 18 (table 4)
caagcagaagacggcatacgagacgttcgaggtgactggagttcagacgtgtgctcttc

SEQ ID NO: 95 - DNA, 59 bp, Artificial Sequence
  Index primer sequence 19 (table 4)
caagcagaagacggcatacgagaaacctagtgtgactggagttcagacgtgtgctcttc

SEQ ID NO: 96 - DNA, 59 bp, Artificial Sequence
  Index primer sequence 20 (table 4)
caagcagaagacggcatacgagagtaggtccgtgactggagttcagacgtgtgctcttc

SEQ ID NO: 97 - DNA, 59 bp, Artificial Sequence
  Index primer sequence 21 (table 4)
caagcagaagacggcatacgagaacggatgggtgactggagttcagacgtgtgctcttc

SEQ ID NO: 98 - DNA, 59 bp, Artificial Sequence
  Index primer sequence 22 (table 4)
caagcagaagacggcatacgagatgaatcacgtgactggagttcagacgtgtgctcttc

SEQ ID NO: 99 - DNA, 59 bp, Artificial Sequence
  Index primer sequence 23 (table 4)
caagcagaagacggcatacgagacactggctgtgactggagttcagacgtgtgctcttc

SEQ ID NO: 100 - DNA, 59 bp, Artificial Sequence
  Index primer sequence 24 (table 4)
caagcagaagacggcatacgagagttccatagtgactggagttcagacgtgtgctcttc

SEQ ID NO: 101 - DNA, 59 bp, Artificial Sequence
  Index primer sequence 25 (table 4)
caagcagaagacggcatacgagattaacagcgtgactggagttcagacgtgtgctcttc

SEQ ID NO: 102 - DNA, 59 bp, Artificial Sequence
  Index primer sequence 26 (table 4)
caagcagaagacggcatacgagacagctccggtgactggagttcagacgtgtgctcttc

SEQ ID NO: 103 - DNA, 59 bp, Artificial Sequence
  Index primer seqeunce 27 (table 4)
caagcagaagacggcatacgagagctggtaagtgactggagttcagacgtgtgctcttc

SEQ ID NO: 104 - DNA, 59 bp, Artificial Sequence
  Index primer sequence 28 (table 4)
caagcagaagacggcatacgagaagctagttgtgactggagttcagacgtgtgctcttc

SEQ ID NO: 105 - DNA, 59 bp, Artificial Sequence
  Index primer sequence 29 (table 4)
caagcagaagacggcatacgagagccgctacgtgactggagttcagacgtgtgctcttc

SEQ ID NO: 106 - DNA, 59 bp, Artificial Sequence
  Index primer sequence 30 (table 4)
caagcagaagacggcatacgagaaggatctggtgactggagttcagacgtgtgctcttc

SEQ ID NO: 107 - DNA, 59 bp, Artificial Sequence
  Index primer sequence 31 (table 4)
caagcagaagacggcatacgagacattaagagtgactggagttcagacgtgtgctcttc

SEQ ID NO: 108 - DNA, 59 bp, Artificial Sequence
  Index primer sequence 32 (table 4)
caagcagaagacggcatacgagattacggctgtgactggagttcagacgtgtgctcttc

SEQ ID NO: 109 - DNA, 59 bp, Artificial Sequence
  Index primer sequence 33 (table 4)
caagcagaagacggcatacgagaatctgtacgtgactggagttcagacgtgtgctcttc

SEQ ID NO: 110 - DNA, 59 bp, Artificial Sequence
  Index primer sequence 34 (table 4)
caagcagaagacggcatacgagatgacactggtgactggagttcagacgtgtgctcttc

SEQ ID NO: 111 - DNA, 59 bp, Artificial Sequence
  Index primer sequnece 35 (table 4)
caagcagaagacggcatacgagaccggtagagtgactggagttcagacgtgtgctcttc

SEQ ID NO: 112 - DNA, 59 bp, Artificial Sequence
  Index primer sequence 36 (table 4)
caagcagaagacggcatacgagagatacgctgtgactggagttcagacgtgtgctcttc

SEQ ID NO: 113 - DNA, 59 bp, Artificial Sequence
  Index primer sequence 37 (table 4)
caagcagaagacggcatacgagaaggaccatgtgactggagttcagacgtgtgctcttc

SEQ ID NO: 114 - DNA, 59 bp, Artificial Sequence
  Index primer sequence 38 (table 4)
caagcagaagacggcatacgagactacaagggtgactggagttcagacgtgtgctcttc

SEQ ID NO: 115 - DNA, 59 bp, Artificial Sequence
  Index primer sequence 39 (table 4)
caagcagaagacggcatacgagagattgtccgtgactggagttcagacgtgtgctcttc

SEQ ID NO: 116 - DNA, 59 bp, Artificial Sequence
  Index primer sequence 40 (table 4)
caagcagaagacggcatacgagatccgtgtagtgactggagttcagacgtgtgctcttc

(19) United States
(12) Patent Application Publication
(10) Pub. No.: US 2021/0181195 A1
(43) Pub. Date: Jun. 17, 2021

(54) METHODS, CONJUGATES AND SYSTEMS

(71) Applicant: IMMUNOVIA AB, Lund (SE)

(72) Inventors: Carl Borrebaeck, Lund (SE); Mattias Brofelth, Lund (SE); Anna Isinger Ekstrand, Hoor (SE); Christer Wingren, Sodra Sandby (SE)

(21) Appl. No.: 16/963,298

(22) PCT Filed: Jan. 29, 2019
(86) PCT No.: PCT/EP2019/052105

§ 371 Date: Jul. 20, 2020

(30) Foreign Application Priority Data
Jan. 30, 2018 (GB) 1801481.1

(51) Int. Cl. G01N 33/564; G01N 33/532

(52) U.S. Cl. CPC G01N 33/564; G01N 33/532

(57) ABSTRACT

The present invention provides methods for detecting and/or quantifying one or more biomarker(s) in a biological sample, as well as conjugates and systems for use in that method. The present invention also provides associated kits and methods of diagnosis and/or prognosis of disease, based on the principles of the methods for detecting and/or quantifying one or more biomarker(s).

### FIELD OF INVENTION

The present invention provides methods for detecting and/or quantifying one or more biomarker(s) in a biological sample, as well as conjugates and systems for use in that method. The present invention also provides associated kits and methods of diagnosis and/or prognosis of disease, based on the methods for detecting and/or quantifying one or more biomarker(s).

### BACKGROUND

The identification of new biomarkers is a key component of medical research, and is particularly relevant to drug discovery and the development of disease diagnostic and prognostic methods (Borrebaeck 2017).

One technology currently used for identifying biomarkers is antibody-based microarrays. Important to the functionality of those microarrays is how the antibodies they utilize are designed, in order to bind to their intended targets. However, when affixed to an array the majority of off-the shelf, readily-available polyclonal and monoclonal antibodies display impaired performance (Haab et al 2001, MacBeath 2002). Additionally, when producing a microarray each antibody needs to be individually produced, purified and dispensed via absorption onto the microarray. This creates logistical problems that increase the complexity of microarray production, which reduces the flexibility of the technology. Also, with increasing demand for up-scaled multiplexity, suitable miniaturized arrays in nanoscale format are technically more challenging to produce and smaller spot size are more vulnerable from surrounding particles like dust (Petersson et al 2014a, Petersson et al 2014b, Petersson et al 2014c).

In addition to microarray technology, solution-based bead arrays which utilise sandwich pairs of antibodies can also be used for identifying biomarkers (de Jager et al 2003, Hodge et al 2004). However, it is very difficult to increase the multiplexity of such systems, because generating sandwich pair antibodies creates logistical challenges (de Jager and Rijkers 2006, Elshal and McCoy 2006, Schwenk et al 2008).

Another key drawback in relation to both microarray technology and solution-based bead arrays is that they rely on fluorescence as a detection signal. However, that only provides relative protein levels, thereby generating fold-changes in expression values and not absolute expression values. This also creates issues in relation to sensitivity, leading to poor detection rates of low abundance proteins.

Alternative solution based systems for identifying biomarkers include Proximity Ligation Assay (PLA), Proximity Extension Assay (PEA), which utilize antibodies conjugated to nucleic acids in a non-specific manner (targeting available primary amines) (Darmanis et al 2011, Fredriksson et al 2002, Lundberg et al 2011). However, the use of non-specific conjugates lead to impaired antibody function and heterogeneous mixes of conjugated antibodies, which impacts specificity and effectiveness.

Accordingly, there is a need for an effective, specific and flexible technology for detecting and/or quantifying biomarkers, which can be scaled up for high-throughput work.

Against this background, the inventors have developed the present invention which overcomes the problems in that prior art by utilizing binding moiety-oligonucleotide conjugates, in which the oligonucleotide moiety comprises an identifier nucleotide sequence which is indicative of the biomarker specificity of the binding moiety. Determining the sequence of the oligonucleotide moiety allows the conjugates to be very effective in identifying detecting and/or quantifying specific biomarkers.

Configurations of the conjugates described herein are particularly advantageous over the prior art because they allow for precise and reproducible control over the ratio of the binding moieties and oligonucleotide moieties in the conjugate, which contributes to the specificity and effectiveness of the technology.

Thus, the invention seeks to provide new methods for detecting biomarkers in biological samples.

### SUMMARY OF THE INVENTION

A first aspect of the invention provides a method of detecting and/or quantifying one or more biomarker(s) in a biological sample, the method comprising the steps of:
(a) providing a biological sample to be tested
(b) contacting biomarkers present in the biological sample with one or more binding moiety-oligonucleotide conjugate(s) to generate biomarker-conjugate complexes, each conjugate comprising (i) a binding moiety having binding specificity for one of the one or more biomarkers and (ii) an oligonucleotide moiety comprising an identifier nucleotide sequence which is indicative of the biomarker specificity of the binding moiety; and
(c) determining the nucleotide sequences of the oligonucleotide moieties in the binding moiety-oligonucleotide conjugates within the biomarker-conjugate complexes generated in step (b),

wherein the nucleotide sequences identified in step (c) are indicative of the presence and/or amount of the one or more biomarker(s) of interest in the biological sample.

The concept of biomarkers, and the valuable information they can convey, is readily understood by persons of skill in the art. Thus, by “biomarker” we include any naturally-occurring biological molecule, or component or fragment thereof, the measurement of which can provide information of value in determining the health status of an individual (such as the presence and/or stage of a disease, risk of developing a disease, responsive to therapy, and the like). In one embodiment, the biomarker(s) is/are selected from the list consisting or comprising of: a peptide; a protein; a carbohydrate; a nucleic acid; a lipid; and a small molecule. For example, the biomarker may be the protein or a polypeptide fragment or carbohydrate moiety thereof. Alternatively, the biomarker may be a nucleic acid molecule, for example a deoxyribonucleic acid (DNA) molecule or derivative thereof (such as circulating tumour DNA, ctDNA) or a ribonucleic acid (RNA) molecule or derivative thereof (such as messenger RNA, mRNA, or microRNA, miRNA). Typically, the nucleic acid encodes a protein or part thereof, or is otherwise involved in regulating gene expression.

It will be appreciated by skilled persons that the methods of the invention are suitable for detecting or quantifying a single biomarker in a biological sample or simultaneously detecting or quantifying a plurality of such biomarkers (i.e. multiplex biomarker analysis). Thus, the method may be used to detect or quantify at least 2 biomarkers in a sample, for example 3, 4, 5, 10, 15, 20, 25, 30, 40, 50 75, 100, 150, 200, 300, 400, 500, 1000 or more biomarkers.

By “detecting one or more biomarker(s)”, we include identifying whether any of the one or more biomarker(s) of interest are present in or absent from the biological sample.

It will be appreciated by skilled persons that the methods of the invention comprise indirect detection of the biomarkers, in the sense that presence of the biomarkers is indicated by determining the nucleotide sequences of the oligonucleotide moieties in the binding moiety-oligonucleotide conjugates within the biomarker-conjugate complexes generated in step (b). Accordingly, by “the nucleotide sequences identified in step (c) are indicative of the presence of the one or more biomarker(s)”, we include that if a nucleotide sequence is identified in step (c) which comprises an identifier nucleotide sequence, then detection of that nucleotide sequence indicates or demonstrates that the particular biomarker of which that identifier nucleotide sequence is indicative is present in the biological sample.

For example, detection in step (c) of a PCR amplification product comprising or consisting of the identifier nucleotide sequence indicates the presence of corresponding biomarker in the biological sample.

Similarly, by “quantifying one or more biomarker(s)”, we include identifying the amount or abundance (either in absolute or relative terms) of any of the one or more biomarker(s) of interest in the biological sample. Accordingly, by “the nucleotide sequences identified in step (c) are indicative of the amount of the one or more biomarker(s)”, we include that if a nucleotide sequence is identified which comprises an identifier nucleotide sequence, then the quantity of those nucleotides sequences indicates or demonstrates the amount or abundance of the biomarkers present in the biological sample, for which that identifier nucleotide sequence is indicative. For example, the absolute or relative amount of a PCR amplification product comprising or consisting of the identifier nucleotide sequence in step (c) indicates the amount or abundance of the of corresponding biomarker in the biological sample.

An initial step in the methods of the invention is the provision of a biological sample to be tested, which may be directly obtained from an individual or may be a derivative or extract from such a ‘raw’ sample.

In one embodiment, the biological sample in step (a) comprises or consists of a tissue sample and/or a fluid sample, or an extract therefrom or derivative thereof.

By “tissue sample”, we include a plurality of cells that have the same (or a related function) in a subject or a plurality of cells that have the same origin (that often form part of an organ), which has been removed from a subject. Examples of tissues from which a tissue sample can be taken include: connective tissue; muscle tissue; nervous tissue; and epithelial tissue. In a particular embodiment, the tissue sample can be one or more from the list consisting or comprising of: a biopsy; a tissue swab; and a tissue scraping. In another embodiment, the tissue sample can comprise one or more different types of tissue; for example, two or more different types of tissue; three or more different types of tissue; four or more different types of tissue; five or more different types of tissue; or six or more different types of tissue. It would be known to one skilled in medicine how to provide a tissue sample, as well as how to provide an extract from a tissue sample. For example, an extract from a tissue can be provided by dissociating the cells of the tissue, which can be mediated by physical disruption of the tissue, such as by shaking. Additionally, an extract from a tissue can also comprise lysed cells from the tissue. Cell lysis can be undertaken by chemical or physical means, using methods known in the art.

By “fluid sample”, we include a liquid that has a biological origin. By “bodily fluid”, we include a liquid that is directly obtainable from the body. As will be appreciated, such a liquid can comprise a number of biological molecules that have the potential to serve as biomarkers. Methods for collecting a fluid sample and/or a bodily fluid are well known to one skilled in medicine, as are methods for obtaining an extract or derivative of such as sample. For example, providing an extract from a fluid sample may comprise isolating a particular fraction of the fluid sample or a particular type of cell(s) from the fluid sample. A specific example of an extract from a fluid sample (in particular, an extract from a bodily fluid sample) is blood plasma, which can be isolated from whole blood via centrifugation.

In one embodiment, the sample is selected from the list consisting or comprising of: blood; serum, plasma, urine; cells (including cell line cultures), tissue, faeces; synovial fluid; saliva; amniotic fluid; endolymph; cerebrospinal fluid; pericardial fluid; pus; gastric fluid; and vomit. In a particular embodiment, the blood consists or comprises of one or more from the list consisting or comprising of: whole blood, blood plasma; leukocytes; erythrocytes; and platelets.

In one embodiment, the sample has a volume of about 1 millilitre (ml) or less; for example: about 950 microlitres (μl) or less; about 900 μl or less; about 850 μl or less about 800 μl or less; about 750 μl or less; about 700 μl or less; about 650 μl or less; about 600 μl or less; about 550 μl or less; about 500 μl or less; about 450 μl or less; about 400 μl or less; about 350 μl or less; about 300 μl or less; about 250 μl or less; about 200 μl or less; about 150 μl or less; about 100 μl or less; about 50 μl or less; about; 10 μl or less; about 9 μl or less; about 8 μl or less; about 7 μl or less; about 6 μl or less; about 5 μl or less; about 4 μl or less; about 3 μl or less; about 2 μl or less; about 1 μl or less; about 900 nanolitres (nl) or less; about 850 nl or less about 800 nl or less; about 750 nl or less; about 700 nl or less; about 650 nl or less; about 600 nl or less; about 550 nl or less; or about 500 nl or less. In an alternative embodiment, the sample has a volume of about 1 ml to about 500 nl; for example: about 500 μl to about 1 μl; about 400 μl to about 1 μl; about 300 μl to about 1 μl; about 200 μl to about 1 μl; about 100 μl to about 1 μl; about 50 μl to about 1 μl; about 10 μl to about 1 μl; about 9 μl to about 1 μl; about 8 μl to about 1 μl; about 7 μl to about 1 μl; about 6 μl to about 1 μl; about 5 μl to about 1 μl, more preferably about 10 μl to about 1 μl.

In one preferred embodiment, the method further comprises step (a′), following step (a), of immobilising the one or more biomarkers present in the sample on a substrate. A particular advantage of immobilising the biomarkers on a substrate is that it concentrates the biomarkers in a location (i.e. on the substrate), which can further improve the effectiveness and sensitivity of the method. Additionally, immobilising the biomarkers on a substrate allows the biomarkers to be readily separated from other components of the biological sample, which further improves the practicality of the method.

By “immobilising the one or more biomarkers present in the sample on a substrate”, we include any method of physically associating biomarkers present in the sample with a substrate, for example by binding biomarkers present in the sample to a substrate (by covalent and/or non-covalent means).

In one embodiment, the one or more biomarkers are immobilised on the substrate using molecular linkages. For example, the substrate could be carboxylated. Molecular linkages, and associated methods of mediating molecular linkages, suitable for immobilising the one or more biomarkers on the substrate are disclosed in Jonkheijm et al., 2008, Angew. Chem. Int. Ed 47:9618-47 (the disclosures of which are incorporated by reference). In a particular embodiment, the molecular linkages are covalent linkages.

In a preferred embodiment, the molecular linkage comprises a pair of corresponding molecular linkage members that bind to (or associate with) each other, wherein one member of the pair is bound to (or associated with) the one or more biomarker(s) and the other member of the pair is bound to (or associated with) a substrate, and wherein the one or more biomarkers are immobilised on the substrate when the pair of corresponding molecule linkage members are bound to (or associated with) each other. As will be appreciated, the member of the pair of corresponding molecular linkages that is bound to (or associated with) the one or more biomarker(s) may be joined to the biomarker via a non-specific binding (or association), wherein each of the one or more biomarkers in the sample is bound to (or associated with) that one of the pair without the need for further modification.

In a preferred embodiment, step (a′) comprises immobilising biotinylated biomarkers on a streptavidin-coated or avidin-coated substrate. In this embodiment, the biotin and streptavidin (or biotin and avidin) represent the pair of corresponding molecular linkages. The one or more biomarkers can be biotinylated using methods known in the art, such as the EZ-Link Sulfo-NHS-LC-Biotin kit (Pierce, Rockford, Ill., USA) following established protocols (Gerdtsson et al., 2016, Ingvarsson et al., 2007; the disclosures of which are incorporated herein by reference).

Typically, the substrate will be coated (or otherwise admixed) with one member of the molecular linkages. By “coated”, we include that part of the surface or all of the surface of the substrate is covered by the molecular linkage member, e.g. streptavidin or avidin.

In an alternative embodiment, the surface of the substrate may be functionalised, wherein the functionalised surface of the substrate can be bound by (or associated with) the one or more biomarkers in a non-specific manner. For example, the surface of the substrate may comprise poly(dimethylsiloxane) (PDMS) which can be treated with plasma oxidation, allowing the PDMS to be functionalised with organosilanes to provide a surface that can bind protein biomarkers in a non-specific manner.

The substrate may comprise or consist of any suitable material, such as a plastic (such as a polymer) and/or a metal and/or glass. For example, the substrate may comprise or consist of a polymer selected from the list consisting of: cellulose; polyacrylamide; nylon; polystyrene; polyvinyl chloride; and polypropylene.

In a further embodiment, the substrate form is selected from the list consisting: particles (such as beads) and planar surfaces (such as array plates).

Suitable particles, such as those with a spherical or bead structure, are well known in the art. For example, the particles may be polymer beads. In a preferred embodiment, the substrate comprises of consists of superparamagnetic polymer beads. An example of such superparamagnetic polymer beads is Dynabeads® (e.g. M-280, MyOne T1, M-270, MyOne C1, available from Life Technologies, CA, USA).

Arrays per se are also well known in the art. Typically, they are formed of a linear or two-dimensional structure having spaced apart (i.e. discrete) regions (“spots”), each having a finite area, formed on the surface of a solid support. Typically, the array is a microarray. By “microarray” we include the meaning of an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. The array may also be a macroarray or a nanoarray. Examples of array formats are described in Steinhauer et al., Biotechniques, 2002 Suppl:38-45; Wingren and Borrebaeck, 2008, Curr Opin Biotechnol; 19:55-61; Wingren et al., Proteomics, 2005; 5:1281-91, Delfani et al., 2016, PLoS One; 11:e0159138 (the disclosures of which are incorporated herein by reference).

Step (b) of the methods of the invention comprises contacting biomarkers present in the biological sample with one or more binding moiety-oligonucleotide conjugates to generate biomarker-conjugate complexes, each conjugate comprising (i) a binding moiety having binding specificity for one of the one or more biomarkers and (ii) an oligonucleotide moiety comprising an identifier nucleotide sequence which is indicative of the biomarker specificity of the binding moiety.

The binding moiety-oligonucleotide conjugates may be as described below in relation to the second aspect of the invention, for example antibody-oligonucleotide conjugates such as scFv-oligo conjugates.

By “contacting biomarkers present in the biological sample with one or more binding moiety-oligonucleotide conjugate(s) to generate biomarker-conjugate complexes”, we include that the step allows for the biomarkers in the biological sample to bind to (or associate with) the binding moieties of the binding moiety-oligonucleotide conjugates.

By “binding moiety having binding specificity for one of the one or more biomarkers”, we include that the binding moiety is able to bind specifically to (or associate with) one of the biomarkers of interest.

By “an identifier nucleotide sequence which is indicative of the biomarker specificity of the binding moiety”, we include that the specific sequence of nucleotides in the identifier nucleotide sequence permits the unambiguous identification of the specific binding target (i.e. the biomarker) of the binding moiety. This allows for the biomarker bound by the binding moiety to be identified indirectly by the sequence of nucleotides of the identifier nucleotide sequence. The identifier nucleotide sequence is therefore analogous to a barcode, as discussed in the Example.

Accordingly, in one embodiment each binding moiety-oligonucleotide conjugate that generates biomarker-conjugate complexes with a particular biomarker comprises the same identifier nucleotide sequence, which is different to the identifier nucleotide sequences of binding moiety-oligonucleotide conjugates that generate biomarker-conjugate complexes with other, different biomarkers. For example, the binding moiety-oligonucleotide conjugates that comprise the same (i.e. a common) binding moiety may also comprise the same identifier nucleotide sequence, which is different to the identifier nucleotide sequences of binding moiety-oligonucleotide conjugates comprising different binding moieties. By “same identifier nucleotide sequence”, we include that the identifier nucleotide sequences all comprise an identical sequence of nucleotides.

In one embodiment, contacting biomarkers present in the biological sample with one or more binding moiety-oligonucleotide conjugate(s) comprises adding the one or more binding moiety-oligonucleotide conjugate(s) to the biological sample. In an alternative embodiment, contacting biomarkers present in the biological sample with one or more binding moiety-oligonucleotide conjugate(s) comprises adding the biological sample to the one or more binding moiety-oligonucleotide(s), such as adding the biological sample to a solution comprising the one or more binding moiety-oligonucleotide(s).

Where the biomarkers are immobilised to a substrate, the method may further comprise step (a″), following step (a′), of separating from the biological sample the immobilised biomarkers bound to the substrate. In a particular embodiment, step (a″) comprises moving the substrate to a new solution or reaction vessel.

Conveniently, step (b) is performed in a solution, under conditions which enable the binding moiety-oligonucleotide conjugates (optionally immobilised to a particulate substrate, e.g. beads) to bind specifically to the biomarker(s) from the biological sample to which they are targeted.

By “conditions which enable the binding moiety-oligonucleotide conjugates to bind specifically to the biomarker(s) to which they are targeted”, we include an environment (for example, a solution) which is conducive for the specific binding (or associating) of the binding moiety-oligonucleotide conjugates to the biomarkers, with no or only negligible non-specific binding (e.g. to other components or molecules present in the sample).

In one embodiment, the method further comprises step (b′), following step (b) and prior to step (c), of removing unbound binding moiety-oligonucleotide conjugates. By “unbound binding moiety-oligonucleotide conjugates”, we include binding moiety-oligonucleotide conjugates that are not bound to (or associated with) biomarkers. It would be known to one skilled in biology and/or chemistry how to remove unbound binding moiety-oligonucleotide conjugates. For example, this could be done using any method of separation, such as solid phase extraction and/or size exclusion (in particular, if a substrate is not used as part of the method).

Accordingly, in one embodiment step (b′) further comprises removing unbound binding moiety-oligonucleotide conjugates by solid phase extraction and/or size exclusion. In a particular embodiment, the size exclusion is size exclusion chromatography, and binding moiety-oligonucleotide conjugates of about 30 kDa or less are removed; for example: about 35 kDa or less; about 40 kDa or less; about 45 kDa or less; about 55 kDa or less; about 60 kDa or less; about 65 kDa or less; or about 70 kDa or less, more preferably about 60 kDa or less.

Alternatively, where the method comprises immobilising the biomarkers on a substrate, step (b′) may comprise washing and/or physically moving the substrate on which are biomarker-conjugate complexes, to remove or separate the complexes from unbound binding moiety-oligonucleotide conjugates.

The binding moiety-oligonucleotide conjugates used in step (b) typically comprise (i) a binding moiety having binding specificity for one of the one or more biomarkers and (ii) an oligonucleotide moiety comprising an identifier nucleotide sequence which is indicative of the biomarker specificity of the binding moiety, thus enabling the conjugate to bind directly to a biomarker of interest.

However, for an analysis with a limited number of targets (e.g. below 20 biomarkers), it will be appreciated by persons skilled in that art that the method can be modified with sandwich pairs; in this embodiment, a primary binding moiety (e.g. an antibody or antigen-binding fragment thereof) is used to bind the biomarkers from the biological sample and then a secondary binding moiety-oligonucleotide conjugate (e.g. an scFv-oligo) is used in which the binding moiety is targeted to the primary binding moiety. Thus, in an alternative embodiment, the binding moiety-oligonucleotide conjugates may bind indirectly to the biomarkers of interest via a primary binding moiety.

Step (c) of the methods of the invention comprises determining the nucleotide sequences of the oligonucleotide moieties in the binding moiety-oligonucleotide conjugates within the biomarker-conjugate complexes generated in step (b).

By “determining the nucleotide sequences of the oligonucleotide moieties in the binding moiety-oligonucleotide conjugates within the biomarker-conjugate complexes”, we include identifying the sequence of nucleotides of the oligonucleotide moieties in binding moiety-oligonucleotide conjugates that are bound to (or associated with) biomarkers. Accordingly, the sequence of nucleotides of the oligonucleotide moieties in binding moiety-oligonucleotide conjugates that are not bound to (or not associated with) biomarkers are excluded. As the oligonucleotide moieties comprise the identifier nucleotide sequence, “determining the nucleotide sequences of the oligonucleotide moieties in the binding moiety-oligonucleotide conjugates within the biomarker-conjugate complexes” includes determining the nucleotide sequences of the identifier nucleotide sequences.

In one embodiment, step (c) comprises determining the nucleotide sequences of the oligonucleotide moieties within the binding moiety-oligonucleotide conjugates by DNA sequencing and/or RNA sequencing.

In one embodiment, the DNA sequencing and/or RNA sequencing comprises high-throughput nucleic acid sequencing, for example using next generation nucleic acid sequencing methods (or NGS).

The next generation nucleic acid sequencing method may be selected from the list consisting of: real time sequencing (such as single molecule real time sequencing); pyrosequencing; Solexa sequencing; sequencing by ligation (such as SOLiD sequencing); Ion Torrent semiconductor sequencing; high-throughput sequencing systems such as MiSeq, HiSeq 2500, and/or NextSeq 500 sequencing; DNA nanoball sequencing; Nanostring; Heliscope single molecule sequencing; and nanopore sequencing. The aforementioned next generation nucleic acid sequencing techniques would be known to one skilled in biology. Real time sequencing can be undertaken using, for example, technology developed by Oxford Nanopore Technologies (Oxford, UK), such as the MinION nanopore DNA sequencer. Pyrosequencing can be undertaken using, for example, technology developed by Qiagen (Venlo, Holland), such as the PyroMark Q24 range of sequencers. Solexa sequencing can be undertaken using, for example, technology developed by IIlumina (San Diego, Calif., USA). Sequencing by ligation (such as SOLiD sequencing) can be undertaken using, for example, technology developed by Life Technologies (California, USA). Ion Torrent semiconductor sequencing can be undertaken using, for example, technology developed by ThermoFisher Scientific (Waltham, Mass., USA). HiSeq 2500 sequencing can be undertaken using, for example, technology developed by Illumina (San Diego, Calif., USA). DNA nanoball sequencing can be undertaken using, for example, technology developed by Complete Genomics (Mountain View, Calif., USA). Nanostring can be undertaken using, for example, technology developed by Nanostring technologies (Washington, USA). Heliscope single molecule sequencing can be undertaken using, for example, technology developed by Helicos BioSciences (Cambridge, Mass., USA). Nanopore sequencing can be undertaken using, for example, technology developed by Oxford Nanopore Technologies (Oxford, UK).

In one embodiment, the method further comprises step (d) of analysing the nucleic acid sequences identified in step (c) to categorise the biological sample. For example, the sequence information obtained in step (c) may enable a biomarker signature to be identified for the sample, which may indicate a disease state in the subject from whom the sample was obtained (see below).

Conveniently, analysis of the nucleic acid sequences identified in step (c) is performed using a support vector machine (SVM), such as those available from http://cran.r-project.org/web/packages/e1071/index.html (e.g. e1071 1.5-24). However, any other suitable data analysis means may also be used.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training data points of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using biomarker profiles from individuals with known disease status (for example, individuals known to have pancreatic cancer). By running such training samples, the SVM is able to learn what biomarker profiles are associated with a particular disease state, such as pancreatic cancer. Once the training process is complete, the SVM is then able to determine whether or not the biomarker sample tested is from an individual with that disease state. Alternatively, this training procedure can be by-passed by pre-programming the SVM with the necessary training parameters.

In one embodiment, step (d) comprises removing or eliminating contaminated and/or mismatched sequences.

In a further embodiment, step (d) comprises removing or eliminating sequence bias from the data. For example, the random sequences present in the oligonucleotide sequences of the conjugates may be used to detect such bias.

Typically, the method further comprises determining the presence and/or quantity of the biomarkers in one or more positive and/or negative control samples alongside the biomarkers from the biological sample.

By “positive control sample”, we include a sample in which it is known that the one or more biomarker(s) of interest is present. For example, the positive control sample could be a biological sample from a subject that is known to have the one or more biomarker(s) (for example, a subject with a disease that is known to be characterised by the one of more biomarker(s)). Alternatively, the positive control sample could an artificial sample or a biological sample to which the one or more biomarker(s) have been added.

By “negative control sample”, we include a sample in which it is known that the one or more biomarker(s) of interest is not present. For example, the negative control sample could be a biological sample from a subject that is known not to have the one or more biomarker(s) (for example, a subject from a species known not to have the one or more biomarker(s)). Alternatively, the positive control sample could be an artificial sample to which the one or more biomarker(s) has not been added.

It would be well known to one skilled in biology that controls (such as positive and/or negative control samples) can be useful in scientific methods. For example, controls can be used to calibrate equipment and/or provide confidence that the results are accurate, and do not include false positive results or false negative results.

By “determining the presence and/or quantity of the biomarkers in one or more positive and/or negative control samples alongside the biomarkers from the biological sample”, we include that the same method steps undertaken on the biological sample are also undertaken on the positive control sample and/or the negative control sample. Specifically, by “alongside” we include that the method is undertaken on the biological sample and the positive control sample and/or negative control sample during the same period of time and/or using the same reagents and/or using the same equipment, to allow for the control samples be used to identify any potential discrepancies in the method. When the outcome of the methods undertaken on the biological sample and the positive control sample and/or negative control sample is known they can be compared. If the nucleotide sequences identified in step (c) are indicative of the presence and/or amount of the one or more biomarker(s) of interest in the positive control sample, it is a good indication that the method has been undertaken correctly. However, if the nucleotide sequences identified in step (c) are indicative of the presence and/or amount of the one or more biomarker(s) of interest in the negative control sample, it is an indication that there has been an error in the methodology and the method might need to be repeated.

In one embodiment, the method comprises or consists multiplex protein analysis in a solution. Thus, the method may permit the simultaneous detection and/or quantification of a plurality of biomarkers in a liquid biological sample, such as plasma or serum.

It will be appreciated by skilled persons that the methods described herein are performed in vitro.

In one preferred embodiment of the invention, the methods are predominantly undertaken in a solution or liquid, in particular steps (b) and/or (c) and/or (d) are undertaken in a solution or a liquid (and sub-steps, such as step (b′), therein). This feature clearly differentiates the methods described herein from some array-like technologies, for example in which antibodies are affixed to a planar array for detection of biomarkers.

A second, related aspect of the invention provides a binding moiety-oligonucleotide conjugate for use in a method according to the first aspect of the invention,
wherein the binding moiety-oligonucleotide conjugate comprises (i) a binding moiety having binding specificity for a biomarker and (ii) an oligonucleotide moiety comprising an identifier nucleotide sequence which is indicative of the binding specificity of the binding moiety;
wherein the conjugation of the oligonucleotide moiety to the binding moiety is site-specific at a connection position on the binding moiety; and
wherein the binding moiety comprises a single connection position.

By “site-specific” conjugation we include that the binding moiety is connected to the oligonucleotide moiety at a unique site, for example at a particular amino acid sequence within the binding moiety. Alternative conjugation methods, for example leading to conjugation at any free amine moiety within a binding moiety, are not considered site-specific.

A particular advantage of the configuration of the binding moiety-oligonucleotide conjugates of the invention is that the site-specific conjugation of the oligonucleotide moiety to the binding moiety at a connection position allows for a precise, and reproducible, control of formation of the conjugates, which contributes to the specificity and effectiveness of the technology and avoids the function of the binding moiety being impaired.

In a preferred embodiment of the second aspect of the invention, the connection position is a single connection position.

It will be appreciated by persons skilled in the art that the conjugate may comprise a ratio of one binding moiety to one or more oligonucleotide moiety; for example, one binding moiety to two or more oligonucleotide moieties; one binding moiety to three or more oligonucleotide moieties; one binding moiety to four or more oligonucleotide moieties; or one binding moiety to five or more oligonucleotide moieties. Advantageously, however, the ratio in each conjugate is one binding moiety to one oligonucleotide moiety.

Suitable binding moieties for use in the conjugates of the invention can be selected from a library, based on their ability to bind a given target molecule (i.e. biomarker), as discussed below.

In one embodiment, the binding moiety is a polypeptide, such as peptide or protein.

For example, the binding moiety may be selected from the list consisting of: an antibody or antigen-binding fragment thereof; a receptor, an aptamer; an affibody; and a nucleic acid.

Molecular libraries such as antibody libraries (such as the n-CoDeR library of BioInvent International AB; see Söderlind et al., 2000), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

Methods for the production and use of antibodies are well known in the art, for example see Antibodies: A Laboratory Manual, 1988, Harlow & Lane, Cold Spring Harbor Press, ISBN-13: 978-0879693145, Using Antibodies: A Laboratory Manual, 1998, Harlow & Lane, Cold Spring Harbor Press, ISBN-13: 978-0879695446 and Making and Using Antibodies: A Practical Handbook, 2006, Howard & Kaser, CRC Press, ISBN-13: 978-0849335280 (the disclosures of which are incorporated herein by reference).

An antigen-binding fragment may comprise one or more of the variable heavy (V_{H}) or variable light (V_{L}) domains. For example, the term antibody fragment includes Fab-like molecules (Better et al (1988) Science 240, 1041); Fv molecules (Skerra et al (1988) Science 240, 1038); single-chain Fv (scFv) molecules where the VH and VL partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544).

The term “antibody variant” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

Aptamers are oligonucleotide or peptide molecules that bind to a specific target molecule (i.e. a biomarker). Oligonucleotide aptamers can be identified and selected using systematic evolution of ligands by exponential enrichment (SELEX). Peptide molecule aptamers comprise one or more peptide loops of variable sequence displayed by a protein scaffold. They are typically isolated from combinatorial libraries and often subsequently improved by directed mutation or rounds of variable region mutagenesis and selection.

Affibody molecules are peptides or proteins engineered to bind to a number of targets (i.e. biomarkers) with high affinity, imitating monoclonal antibodies, and are therefore a member of the family of antibody mimetics.

Receptors are typically proteins that function in cellular signal transduction. Receptors will often bind (or associate) with one or more molecules (usually referred to as ligands) during normal function. For example, cytokines have a corresponding cytokine receptor to which they bind. The binding of a receptor to a ligand can be utilised as part of this invention, in which case the receptors one or more ligands may be the one or more biomarker(s) of interest.

In one embodiment, the antibody is of an isotype selected from the list consisting or comprising of: IgG; IgA; IgD; IgE and IgM. For example, the antibody may be an IgG molecule. One skilled in biology would be familiar with antibody isotypes, such as those discussed herein.

In one embodiment, the antibody is selected from the list consisting of: a mammalian antibody; and chimeric (e.g. humanised) antibody. In a particular embodiment, the mammalian antibody is an antibody from a mammal selected from the list consisting or comprising of: a rodent (for example, a mouse, and/or a rat, and/or a hamster, and/or a guinea pig, and/or a gerbil, and/or a rabbit); a canine (for example, a dog); a feline (for example, a cat); a primate (for example, a human; and/or a monkey; and/or an ape); an equine (for example, a horse); a bovine (for example, a cow); and a porcine (for example, a pig). In a preferred embodiment, the mammalian antibody is an antibody from a mammal selected from the list consisting of: a human, a mouse and a rabbit. A humanised antibody is an antibody from a non-human species which has been modified to resemble a human antibody. An example of such a modification is to exchange the fragment crystallisable region (Fc region) of a non-human antibody for the Fc region of a human antibody.

Thus, the binding moiety may be an intact antibody.

Alternatively, the binding moiety may be an antigen-binding fragment (or antigen-binding derivative of an antibody) selected from the group consisting or comprising of: a single-chain Fv (scFv, including multimers thereof such as diabodies, triabodies, minibodies, and the like); a Fab fragment; a F(ab′) fragment (such as F(ab′)2 and Fab′); a disulfide-linked Fv (sdFv); and a single domain antibody. In a preferred embodiment, the binding moiety is an scFv. These particular antigen-binding fragments are defined further herein, and/or would be known to one skilled in biology.

In one embodiment, the antibody or fragment thereof is a monoclonal antibody or fragment (or derivative).

Conveniently, the antibody or fragment (or derivative) thereof is a recombinant antibody or fragment.

Recombinant antibodies are produced through recombination of different nucleotide sequences to produce an antibody which can comprises features from different genetic sources, such as different subjects and/or species. For example, a recombinant antibody can comprise different V_{H} and V_{L} domains, and be produced by those domains being cloned into, and expressed from, a high-yield expression vector.

It will be appreciated that the conjugation of the oligonucleotide moiety to the binding moiety may be direct or indirect.

By the conjugation being “direct”, we include that the connection position(s) of the binding moiety (for example, if the binding moiety is an antibody then one or more amino acids of the binding moiety, at the connection position) is conjugated directly to one or more nucleotides of the oligonucleotide moiety. For example, such a direct conjugation could be mediated by one or more amino acids and/or one or more nucleotides, which are capable of forming, or have been modified in order to form, covalent bonds.

By the conjugation being “indirect”, we include that the conjugate comprises one or more additional components which are not integral to either the binding moiety and/or the oligonucleotide moiety, but which mediate the conjugation at the connection point. Accordingly, in one embodiment, the conjugate comprises one or more means for conjugating the binding moiety to the oligonucleotide moiety, at the connection position; for example, two or more means; three or more means; four or more means; and five or more means. The means for conjugating the binding moiety to the oligonucleotide moiety can be attached or otherwise joined to either the binding moiety or the oligonucleotide moiety, or both the binding moiety and the oligonucleotide moiety, prior to the binding moiety being indirectly conjugated to the oligonucleotide moiety. Thus, the binding moiety and/or the oligonucleotide moiety may comprise the means for conjugating the binding moiety to the oligonucleotide moiety.

Strategies suitable for “indirect” conjugation between a binding moiety (in particular a polypeptide, such as an antibody) and an oligonucleotide moiety are well known to one skilled in chemistry. For example, one strategy for indirect conjugation is sortase-mediated conjugation). The transpeptidase sortase A has a well-known history of being used for post-translational labelling of several types of proteins (Guimaraes et al 2013, Parthasarathy et al 2007). Upon recognition of the LPETG-motif (designed within the acceptor molecule), sortase A cleaves between the Thr and Gly and forms a thioester intermediate between the engineered molecules. Binding moieties can be designed to include the acceptor molecule (scFv-Srt-His6) and the oligonucleotides, carrying as tri-glycine in their 5′-end as the molecule to be attached (see further details below).

Thus, conveniently, the binding moiety is a polypeptide and the connection position comprises or consists of one or more amino acids fused to or otherwise within the binding moiety. For example, the means for conjugating the binding moiety to the oligonucleotide moiety may comprise or consist of one or more amino acids; e.g. two or more; three or more; four or more; five or more; six or more; seven or more; eight or more; nine or more; 10 or more; 11 or more; 12 or more; 13 or more; 14 or more; 15 or more; 16 or more; 17 or more; 18 or more; 19 or more; 20 or more; 25 or more; 30 or more; 35 or more; 40 or more; 45 or more; or 50 or more amino acids, preferably three or more amino acids. It will be appreciated that any type of amino acids may be used, such as naturally occurring amino acids (e.g. L-amino acids).

In one embodiment, the binding moiety is a polypeptide and the connection position is at the N-terminus of the binding moiety and/or at the C-terminus of the binding moiety, preferably at the N-terminus of the binding moiety.

In a further embodiment, the connection position of the binding moiety is directly or indirectly conjugated to the oligonucleotide moiety at the 5′ terminus of the oligonucleotide moiety and/or the 3′ terminus of the oligonucleotide moiety, more preferably at the 5′ terminus of the oligonucleotide moiety.

In one embodiment, the connection position comprises or consists of a sortase tag.

For example, binding moieties can be designed to include the acceptor sequence (e.g. LPETG) capable of conjugating with oligonucleotides carrying a tri-glycine in their 5′-end.

Thus, in one embodiment, the binding moiety within the conjugate is a polypeptide wherein the connection position comprises or consists of the amino acid sequence LPXTG, wherein X can be any amino acid (preferably LPETG). For example, the binding moiety may be a polypeptide wherein the connection position comprises or consists of the amino acid sequence (GS)ₙLPXTGₘ, wherein n is an integer between 1 and 6 and m is an integer between 1 and 6. Advantageously, the binding moiety is a polypeptide and the connection position comprises or consists of the amino acid sequence (GS)₃LPXTG₃.

In an alternative embodiment, the binding moiety is conjugated to the oligonucleotide moiety by thiol-maleimide conjugation.

Thus, the binding moiety may be a polypeptide wherein the connection position comprises or consists of the amino acid sequence (His)ₙ, wherein n is an integer between 1 and 10 (preferably n=6).

A further alternative strategy by which to conjugate the binding moiety and the oligonucleotide moiety is “click chemistry” (Kim et al 2013, Nienberg et al 2016). Using click chemistry binding pairs of molecules, such as small molecules, can be conjugated to the connection position of the binding moiety and the oligonucleotide moiety, and it is the click chemistry molecules that conjugate together to indirectly conjugate the binding moiety and the oligonucleotide moiety. Thus, in one embodiment, the means for conjugating the binding moiety to the oligonucleotide moiety comprises or consists of a binding pair of molecules for click chemistry, wherein the binding moiety comprises one of the binding pair of molecules for click chemistry and the oligonucleotide moiety comprises the corresponding member of the binding pair for click chemistry. For example, the binding pair of molecules for click chemistry may be selected from the following:
(a) an azide-group and an alkyne group (which can be catalyzed by Cu(I) to generate a triazole crosslink between the binding moiety and the oligonucleotide moiety);
(b) an azide-group and an dibenzymcyclooctyne (DBCO) group (which can also generate a triazole crosslink between the binding moiety and the oligonucleotide moiety); and
(c) an tetrazine-group and an alkene group, such as a vinyl, trans-cyclooctene or methylcyclopropene group (which can be catalyzed by Cu(I) to generate a dihydropyrazine crosslink between the binding moiety and the oligonucleotide moiety).

In one such embodiment, the binding pair of molecules for click chemistry comprises an unnatural amino acid (UAA) with a suitable functional group such as an azide group.

The oligonucleotide moiety within the conjugate may be selected from the list consisting of: DNA; RNA; morpholino; peptide nucleic acid (PNA); locked nucleic acid (LNA); glycol nucleic acid (GNA); threose nucleic acid (TNA); and derivatives thereof. In a preferred embodiment, the oligonucleotide moiety comprises or consists of DNA.

In one embodiment, the oligonucleotide moiety comprises about 10 or more nucleotides in length; for example: about 20 or more nucleotides; about 30 or more nucleotides; about 40 or more nucleotides; about 50 or more nucleotides; about 60 or more nucleotides; about 70 or more nucleotides; about 80 or more nucleotides; about 90 or more nucleotides; or about 100 or more nucleotides in length, more preferably about 61 or more nucleotides; about 62 or more nucleotides; about 63 or more nucleotides; about 64 or more nucleotides; about 65 or more nucleotides; about 66 or more nucleotides; about 67 or more nucleotides; about 68 or more nucleotides; or about 69 or more nucleotides in length, even more preferably about 66 nucleotides in length. In a particular embodiment, the oligonucleotide moiety comprises about 50 to about 100 nucleotides in length; for example: about 50 to about 90 nucleotides; about 50 to about 80 nucleotides; about 50 to about 70 nucleotides; about 50 to about 70 nucleotides; about 60 to about 90 nucleotides; about 70 to about 90 nucleotides; about 80 to about 90 nucleotides in length, more preferably about 60 to about 85 nucleotides in length.

The length of the identifier nucleotide sequence within the oligonucleotide moiety will depend upon the number of distinct biomarkers that are to be detected or quantified using the methods of the invention. Based on the use of the four conventional nucleotides A, T G and C, an identifier nucleotide sequence of just three nucleotides in length is sufficient to identify 64 different biomarkers (i.e. 4³=4×4×4).

Thus in one embodiment, the identifier nucleotide sequence is about 3 or more nucleotides in length; for example: about 4 or more nucleotides; about 5 or more nucleotides; about 6 or more nucleotides; about 7 or more nucleotides; about 8 or more nucleotides; about 9 or more nucleotides; about 10 or more nucleotides; about 11 or more nucleotides; about 12 or more nucleotides; about 13 or more nucleotides; about 14 or more nucleotides; about 15 or more nucleotides; about 16 or more nucleotides; about 17 or more nucleotides; about 18 or more nucleotides; about 19 or more nucleotides; about 20 or more nucleotides; about 21 or more nucleotides; about 22 or more nucleotides; about 23 or more nucleotides; about 24 or more nucleotides; or about 25 or more nucleotides in length, more preferably about 6 nucleotides in length. In a particular embodiment, the identifier nucleotide sequence is about 3 to about 25 nucleotides in length; for example: about 3 to about 24 nucleotides; about 3 to about 23 nucleotides; about 3 to about 22 nucleotides; about 3 to about 21 nucleotides; about 3 to about 20 nucleotides; about 3 to about 19 nucleotides; about 3 to about 18 nucleotides; about 3 to about 17 nucleotides; about 3 to about 16 nucleotides; about 3 to about 15 nucleotides; about 3 to about 14 nucleotides; about 3 to about 13 nucleotides; about 3 to about 12 nucleotides; about 3 to about 11 nucleotides; about 3 to about 10 nucleotides; about 3 to about 9 nucleotides; about 3 to about 8 nucleotides; about 3 to about 7 nucleotides; about 3 to about 6 nucleotides; about 3 to about 5 nucleotides; about 4 to about 25 nucleotides; about 5 to about 25 nucleotides; about 6 to about 25 nucleotides; about 7 to about 25 nucleotides; about 8 to about 25 nucleotides; about 9 to about 25 nucleotides; about 10 to about 25 nucleotides; about 11 to about 25 nucleotides; about 12 to about 25 nucleotides; about 13 to about 25 nucleotides; about 14 to about 25 nucleotides; about 15 to about 25 nucleotides; about 16 to about 25 nucleotides; about 17 to about 25 nucleotides; about 18 to about 25 nucleotides; about 19 to about 25 nucleotides; about 20 to about 25 nucleotides; about 21 to about 25 nucleotides; about 3 to about 10 nucleotides; about 4 to about 9 nucleotides; about 5 to about 8 nucleotides in length, preferably about 4 to about 15 nucleotides in length.

In one embodiment, the oligonucleotide moiety further comprises a random nucleotide sequence. The “random nucleotide sequence” provides a unique sequence of nucleotides for a specific binding moiety-oligonucleotide conjugate, allowing calculation of unique sequence counts only during the data analysis phase. Accordingly, in aspects of the invention in which there are a plurality (i.e. two or more) binding moiety-oligonucleotide conjugates (such as the method of the first aspect of the invention or the system of the third aspect of the invention), each conjugate will comprise a random nucleotide sequence comprising a different sequence of nucleotides and/or length, regardless of whether the conjugates comprise the same binding moiety and/or comprise a binding moiety with a binding specificity for a particular biomarker.

In one embodiment, the random nucleotide sequence is about 3 or more nucleotides in length; for example: about 4 or more nucleotides; about 5 or more nucleotides; about 6 or more nucleotides; about 7 or more nucleotides; about 8 or more nucleotides; about 9 or more nucleotides; about 10 or more nucleotides; about 11 or more nucleotides; about 12 or more nucleotides; about 13 or more nucleotides; about 14 or more nucleotides; about 15 or more nucleotides; about 16 or more nucleotides; about 17 or more nucleotides; about 18 or more nucleotides; about 19 or more nucleotides; about 20 or more nucleotides; about 21 or more nucleotides; about 22 or more nucleotides; about 23 or more nucleotides; about 24 or more nucleotides; or about 25 or more nucleotides in length, more preferably about 8 nucleotides in length. In a particular embodiment, the random nucleotide sequence is about 3 to about 25 nucleotides in length; for example: about 3 to about 24 nucleotides; about 3 to about 23 nucleotides; about 3 to about 22 nucleotides; about 3 to about 21 nucleotides; about 3 to about 20 nucleotides; about 3 to about 19 nucleotides; about 3 to about 18 nucleotides; about 3 to about 17 nucleotides; about 3 to about 16 nucleotides; about 3 to about 15 nucleotides; about 3 to about 14 nucleotides; about 3 to about 13 nucleotides; about 3 to about 12 nucleotides; about 3 to about 11 nucleotides; about 3 to about 10 nucleotides; about 3 to about 9 nucleotides; about 3 to about 8 nucleotides; about 3 to about 7 nucleotides; about 3 to about 6 nucleotides; about 3 to about 5 nucleotides; about 4 to about 25 nucleotides; about 5 to about 25 nucleotides; about 6 to about 25 nucleotides; about 7 to about 25 nucleotides; about 8 to about 25 nucleotides; about 9 to about 25 nucleotides; about 10 to about 25 nucleotides; about 11 to about 25 nucleotides; about 12 to about 25 nucleotides; about 13 to about 25 nucleotides; about 14 to about 25 nucleotides; about 15 to about 25 nucleotides; about 16 to about 25 nucleotides; about 17 to about 25 nucleotides; about 18 to about 25 nucleotides; about 19 to about 25 nucleotides; about 20 to about 25 nucleotides; about 21 to about 25 nucleotides; about 3 to about 10 nucleotides; about 4 to about 9 nucleotides; about 5 to about 8 nucleotides in length, preferably about 5 to about 12 nucleotides in length.

In a particular embodiment, the identifier nucleotide sequence and the random nucleotide sequence do not have the same sequence of nucleotides.

In one embodiment, the oligonucleotide moiety further comprises one or more adaptor sequences for hybridising to PCR primers; for example, two or more adaptor sequences; three or more adaptor sequences; four or more adaptor sequences; five or more adaptor sequences; six or more adaptor sequences for hybridising to PCR primers. In a particular embodiment, the oligonucleotide moiety further comprises a first adaptor sequence for hybridising to a universal PCR primer and a second adaptor sequence for hybridising to a sample-specific PCR primer.

In a particular embodiment, the identifier nucleotide sequence and the random nucleotide sequence and the one or more adapter sequences do not have the same sequence of nucleotides.

A third aspect of the invention provides a system for use in a method according to the first aspect of the invention, comprising one or more populations of binding moiety-oligonucleotide conjugates according to the second aspect of the invention;
wherein each population of binding moiety-oligonucleotide conjugates comprises a plurality of conjugates with binding specificity for the same biomarker;
wherein each population of binding moiety-oligonucleotide conjugates comprises a plurality of conjugates comprising an identifier nucleotide sequence which is indicative of the biomarker to which the binding moiety has binding specificity; and
wherein each binding moiety in a population is conjugated to the same number of oligonucleotide moieties.

A particular advantage of the configuration of the binding moiety-oligonucleotide conjugates of the third aspect of the invention is that each binding moiety in a population being conjugated to the same number of oligonucleotide moieties allows for each conjugate of that population to comprise the same ratio of binding moieties to oligonucleotides moieties. Having the same ratio of binding moieties to oligonucleotide moieties allows for an entirely predictable number of oligonucleotides to be associated with each biomarker, which increases the accuracy of methods which use the system of the invention.

In one embodiment, the population of binding moiety-oligonucleotide conjugates comprises a plurality of binding moiety-oligonucleotide conjugates, such as two or more binding moiety-oligonucleotide conjugates; for example: about 10 or more binding moiety-oligonucleotide conjugates; about 20 or more binding moiety-oligonucleotide conjugates; about 30 or more binding moiety-oligonucleotide conjugates; about 40 or more binding moiety-oligonucleotide conjugates; about 50 or more binding moiety-oligonucleotide conjugates; about 60 or more binding moiety-oligonucleotide conjugates; about 70 or more binding moiety-oligonucleotide conjugates; about 80 or more binding moiety-oligonucleotide conjugates; about 90 or more binding moiety-oligonucleotide conjugates; about 100 or more binding moiety-oligonucleotide conjugates; about 200 or more binding moiety-oligonucleotide conjugates; about 300 or more binding moiety-oligonucleotide conjugates; about 400 or more binding moiety-oligonucleotide conjugates; about 500 or more binding moiety-oligonucleotide conjugates; about 600 or more binding moiety-oligonucleotide conjugates; about 700 or more binding moiety-oligonucleotide conjugates; about 800 or more binding moiety-oligonucleotide conjugates; about 900 or more binding moiety-oligonucleotide conjugates; about 1,000 or more binding moiety-oligonucleotide conjugates; or about 10,000 or more binding moiety-oligonucleotide conjugates.

In one embodiment, the connection position(s) is at the same location(s) or region(s) on the binding moiety of each conjugate in a population. In a particular embodiment wherein the binding moiety is an antibody or an antigen-binding fragment, the connection position(s) is at the same sequence(s) of amino acids of each conjugate in a population

In one embodiment, each conjugate in a population comprises a binding moiety that binds to (or associates with) the same epitope of the biomarker. In a preferred embodiment, each conjugate in a population comprises the same binding moiety.

In one embodiment, the system comprises two or more populations of binding moiety-oligonucleotide conjugates, wherein the two or more populations of conjugates have binding specificity for different biomarkers; for example: three or more populations of binding moiety-oligonucleotide conjugates; four or more populations of binding moiety-oligonucleotide conjugates; five or more populations of binding moiety-oligonucleotide conjugates; six or more populations of binding moiety-oligonucleotide conjugates; seven or more populations of binding moiety-oligonucleotide conjugates; eight or more populations of binding moiety-oligonucleotide conjugates; nine or more populations of binding moiety-oligonucleotide conjugates; ten or more populations of binding moiety-oligonucleotide conjugates; 20 or more populations of binding moiety-oligonucleotide conjugates; 30 or more populations of binding moiety-oligonucleotide conjugates; 40 or more populations of binding moiety-oligonucleotide conjugates; 50 or more populations of binding moiety-oligonucleotide conjugates; 60 or more populations of binding moiety-oligonucleotide conjugates; 70 or more populations of binding moiety-oligonucleotide conjugates; 80 or more populations of binding moiety-oligonucleotide conjugates; 90 or more populations of binding moiety-oligonucleotide conjugates; 100 or more populations of binding moiety-oligonucleotide conjugates; 200 or more populations of binding moiety-oligonucleotide conjugates; 300 or more populations of binding moiety-oligonucleotide conjugates; 400 or more populations of binding moiety-oligonucleotide conjugates; 500 or more populations of binding moiety-oligonucleotide conjugates; 600 or more populations of binding moiety-oligonucleotide conjugates; 700 or more populations of binding moiety-oligonucleotide conjugates; 800 or more populations of binding moiety-oligonucleotide conjugates; 900 or more populations of binding moiety-oligonucleotide conjugates; or 1,000 or more populations of binding moiety-oligonucleotide conjugates, wherein each population of conjugates has binding specificity for different biomarkers.

In one embodiment, the system comprises two or more populations of binding moiety-oligonucleotide conjugates, wherein the two or more populations of conjugates comprise different binding moieties; for example: three or more populations of binding moiety-oligonucleotide conjugates; four or more populations of binding moiety-oligonucleotide conjugates; five or more populations of binding moiety-oligonucleotide conjugates; six or more populations of binding moiety-oligonucleotide conjugates; seven or more populations of binding moiety-oligonucleotide conjugates; eight or more populations of binding moiety-oligonucleotide conjugates; nine or more populations of binding moiety-oligonucleotide conjugates; ten or more populations of binding moiety-oligonucleotide conjugates; 20 or more populations of binding moiety-oligonucleotide conjugates; 30 or more populations of binding moiety-oligonucleotide conjugates; 40 or more populations of binding moiety-oligonucleotide conjugates; 50 or more populations of binding moiety-oligonucleotide conjugates; 60 or more populations of binding moiety-oligonucleotide conjugates; 70 or more populations of binding moiety-oligonucleotide conjugates; 80 or more populations of binding moiety-oligonucleotide conjugates; 90 or more populations of binding moiety-oligonucleotide conjugates; 100 or more populations of binding moiety-oligonucleotide conjugates; 200 or more populations of binding moiety-oligonucleotide conjugates; 300 or more populations of binding moiety-oligonucleotide conjugates; 400 or more populations of binding moiety-oligonucleotide conjugates; 500 or more populations of binding moiety-oligonucleotide conjugates; 600 or more populations of binding moiety-oligonucleotide conjugates; 700 or more populations of binding moiety-oligonucleotide conjugates; 800 or more populations of binding moiety-oligonucleotide conjugates; 900 or more populations of binding moiety-oligonucleotide conjugates; or 1,000 or more populations of binding moiety-oligonucleotide conjugates, wherein each population of conjugates comprise different binding moieties.

In one embodiment of the systems of the invention, each conjugate within a population comprises one or more connection positions; for example: two or more connection positions; three or more connection positions; four or more connection positions; or five or more connection positions, more preferably a single connection position.

In a further embodiment of the systems of the invention, each of the conjugates within a population comprises a ratio of one binding moiety to one or more oligonucleotide moiety; for example, one binding moiety to two or more oligonucleotide moieties; one binding moiety to three or more oligonucleotide moieties; one binding moiety to four or more oligonucleotide moieties; or one binding moiety to five or more oligonucleotide moieties.

In a further embodiment of the systems of the invention, the conjugates within each population comprise the same number of oligonucleotide moieties as the number of connection positions on the binding moieties, and optionally one oligonucleotide(s) is conjugated to each connection position(s).

In a preferred embodiment, each of the conjugates within a population comprises a ratio of one binding moiety to one oligonucleotide moiety, more preferably wherein the conjugates within a population comprise one binding moiety and one oligonucleotide moiety.

In one embodiment of the third aspect of the invention, the system comprises populations of binding moiety-oligonucleotide conjugates with specificity to biomarkers in a biomarker signature of a disease state (see below).

In another embodiment of the third aspect of the invention, the system further comprises one or more components from the list consisting of:
(a) one or more substrate(s);
(b) means for immobilising biomarkers to the substrate; and/or
(c) a means for detecting and/or quantifying the identifier nucleotide sequences within the oligonucleotide moieties of the binding moiety-oligonucleotide conjugates;

As will be appreciated, the “one or more substrate(s)” can be any one or more substrate(s) described herein, such as those described in respect of the second aspect of the invention. The “means for immobilising biomarkers to the substrate” can be any technology and/or technique and/or equipment described herein that can be used to immobilize the biomarkers to the substrate, such as those described in respect of the second aspect of the invention. In particular, the “means for immobilising biomarkers to the substrate” can be any technology and/or technique and/or equipment described in Jonkheijm et al, 2008, Angew. Chem. Int. Ed 47:9618-47. The “means for detecting and/or quantifying the identifier nucleotide sequences within the oligonucleotide moieties of the binding moiety-oligonucleotide conjugates” can be any technology and/or technique and/or equipment that can be used for nucleic acid sequencing, such as the DNA, RNA, high-throughput and next generation sequencing discussed in respect of the second aspect of the invention.

In a preferred embodiment, the system comprises superparamagnetic polymer particles.

In a preferred embodiment, the system comprises means for biotinylating biomarkers and/or means for coating the substrate with streptavidin or avidin.

In one embodiment, the system comprises PCR primers for amplifying the identifier nucleotide sequences within the oligonucleotide moieties of the binding moiety-oligonucleotide conjugates.

In one embodiment, the system comprises software or an algorithm for analysing nucleotide sequence data and categorising the biological sample. For example, the system may comprise means for programming a support vector machine.

A fourth aspect of the invention provides a kit of parts for manufacturing a binding moiety-oligonucleotide conjugate as described herein (such as the binding moiety-oligonucleotide conjugate of the second aspect of the invention), or the system as described herein (such as the system of the third aspect of the invention), wherein the kit of parts comprises:
(a) one or more binding moieties as described herein (such as in the second and/or third aspects of the invention); and/or
(b) one or more oligonucleotide moieties as described herein (such as in the second and/or third aspects of the invention).

For example, the kit may comprise one or more populations of binding moieties, wherein each population comprises binding moieties with binding specificity for the same biomarker; and/or wherein the binding moieties are the same binding moieties.

In one embodiment, the kit comprises one or more populations of oligonucleotide moieties, wherein each population comprises binding moieties comprising the same identifier nucleotide sequences.

In additional embodiments, the kit further comprises one or more of the following further components:
(a) means for immobilising biomarkers to the substrate (as described in the third aspect of the invention);
(b) a polypeptide having sortase activity, such as Sortase A; and/or
(c) a water-soluble, amine-to-sulfhydryl crosslinker (e.g. sulfo-SMCC).

A fifth aspect of the invention provides a method of diagnosis and/or prognosis of a disease state in a subject, comprising the steps:
(a) providing a biological sample from the subject to be tested; and
(b) detecting and/or quantifying one or more biomarkers(s) of interest in the sample using a method described herein (such as in the first aspect of the invention);

wherein the presence and/or quantity of the one or more biomarkers(s) of interest is indicative of the disease state.

In one embodiment, the disease state is selected from the group consisting or comprising of:
(a) the presence or absence of a disease;
(b) the stage or extent of a disease progression and/or level of disease activity; and
(c) the responsiveness to a therapeutic agent for treating a disease.

In one embodiment, the disease is selected from a list consisting or comprising of: a cancer; an autoimmune disease; a blood disease; an infectious disease; and a genetic disease.

In one embodiment, the cancer is selected from the group consisting or comprising of cancers of the: pancreas; prostate; breast; ovary; lung; GI tract (e.g. colon); skin; liver; kidney; brain; blood; and bone.

For example, where the cancer is of the pancreas, the cancer may be selected from the list consisting or comprising of: adenocarcinoma; adenosquamous carcinoma; signet ring cell carcinoma; hepatoid carcinoma; colloid carcinoma; undifferentiated carcinoma; and undifferentiated carcinomas with osteoclast-like giant cells, more preferably a pancreatic adenocarcinoma, most preferably pancreatic ductal adenocarcinoma, also known as exocrine pancreatic cancer.

In one embodiment, the disease is an autoimmune disease or disorder, for example selected from the group consisting of: SLE; rheumatoid arthritis; ANCA-associated vasculitis; Sjögren syndrome; and systemic sclerosis.

Biomarker signatures suitable the diagnosis or prognosis of disease states are well known in the art.

For example, the methods of the invention may be used to diagnose pancreatic cancer (or the risk thereof) using the IMMRay® PanCan-D biomarker signature of Immunovia AB (Lund, Sweden), comprising the following protein and carbohydrate biomarkers:
Apolipoprotein A1, Aprataxin and PNK-like factor, Calcineurin B homologous protein 1, Calcium/calmodulin-dependent protein kinase type IV, Complement C3, Complement C4, Complement C5, Cyclin-dependent kinase 2, Disks large homolog 1, GTP-binding protein GEM, HADH2 protein, Intercellular adhesion molecule 1, Interferon gamma, Interleukin-13, Interleukin-4, Interleukin-6, Lewis x, Lymphotoxin-alpha, Membrane-associated guanylate kinase, WW and PDZ domain-containing protein 1, Myomesin-2, Plasma protease C1 inhibitor, PR domain zinc finger protein 8, Properdin, Protein kinase C zeta type, Protein-tyrosine kinase 6, Serine/threonine-protein kinase MARK1, Sialyl Lewis x, Vascular endothelial growth factor and Visual system homeobox 2.

Further examples of suitable biomarker signatures are described in the following published patent applications (the disclosures of which are incorporated herein by reference):
PCT/GB2008/001090, PCT/GB2012/050483, PCT/GB2014/053340, PCT/EP2016/072617, PCT/EP2017/061202 (pancreatic cancer)
PCT/GB2011/051673, PCT/EP2017/063852, PCT/EP2017/063855 (systemic lupus erythematosus)
PCT/EP2014/056630 (prostate cancer)
PCT/GB2008/003922, PCT/GB2011/000865, PCT/IB2013/052858, PCT/GB2015/051678 (breast cancer)

In one embodiment, the method further comprises the step of selecting a treatment for the disease, following the diagnosis or prognosis of the disease state.

In one embodiment, the method further comprises the step of administering to the subject an effective treatment for the disease, following the diagnosis or prognosis of the disease state.

When a diagnosis and/or prognosis of a disease is provided, one skilled in medicine would be aware of what treatment could (and/or should) be administered. For example, in one embodiment, the treatment might be one or more selected from list consisting or comprising of: antibiotics; antivirals; antifungals; immunosuppressants; surgery; radiotherapy; a blood transfusion; a bone marrow transplant; chemotherapy; immunotherapy; chemoimmunotherapy; thermochemotherapy and combinations thereof.

In one embodiment, the subject is a mammalian subject or a non-mammalian subject. In a particular embodiment, the mammalian subject is one of more selected from the group comprising or consisting of: a rodent (for example, a mouse, and/or a rat, and/or a hamster, and/or a guinea pig, and/or a gerbil, and/or a rabbit); a canine (for example, a dog); a feline (for example, a cat); a primate (for example, a human; and/or a monkey; and/or an ape); an equine (for example, a horse); a bovine (for example, a cow); and a porcine (for example, a pig). In a preferred embodiment, the mammalian subject is a human.

It will be appreciated that the invention also provides a use of:
the binding moiety-oligonucleotide conjugates described herein (such as in the second and third aspects of the invention); and/or
the system described herein (such as in the third aspect of the invention) for
detecting and/or quantifying one or more biomarker(s) in a biological sample (such as outlined in the method of the first aspect of the invention) and/or;
providing a diagnosis and/or prognosis of a disease state in a subject (such as outlined in the method of the fifth aspect of the invention).

Preferred, non-limiting examples which embody certain aspects of the invention will now be described, with reference to the following figures:

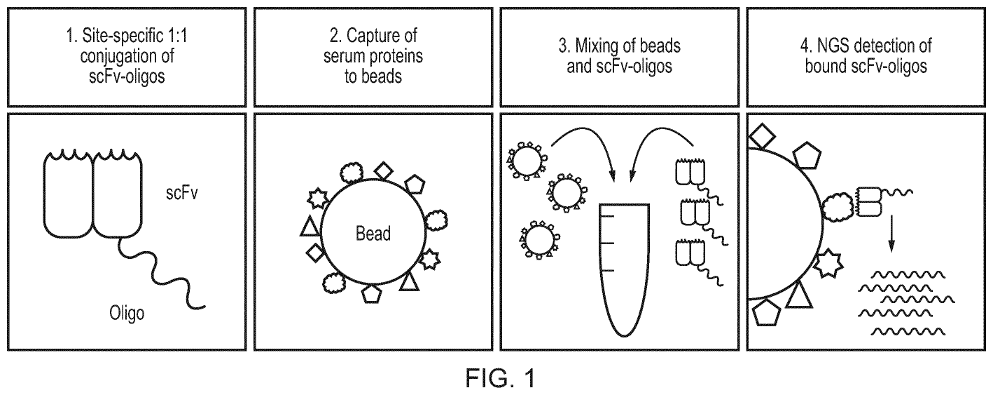

FIG. 1. The concept of MIAS assay. Recombinant scFv antibodies are site-specifically (1:1) conjugated with unique DNA sequences using a Sortase A mediated coupling strategy (1). Biotinylated serum proteins are captured and displayed on magnetic beads (2) and mixed with the DNA-labelled scFv antibodies (3). Unbound scFv antibodies are washed away and bound scFv antibodies are detected and quantified using next generation sequencing (NGS).

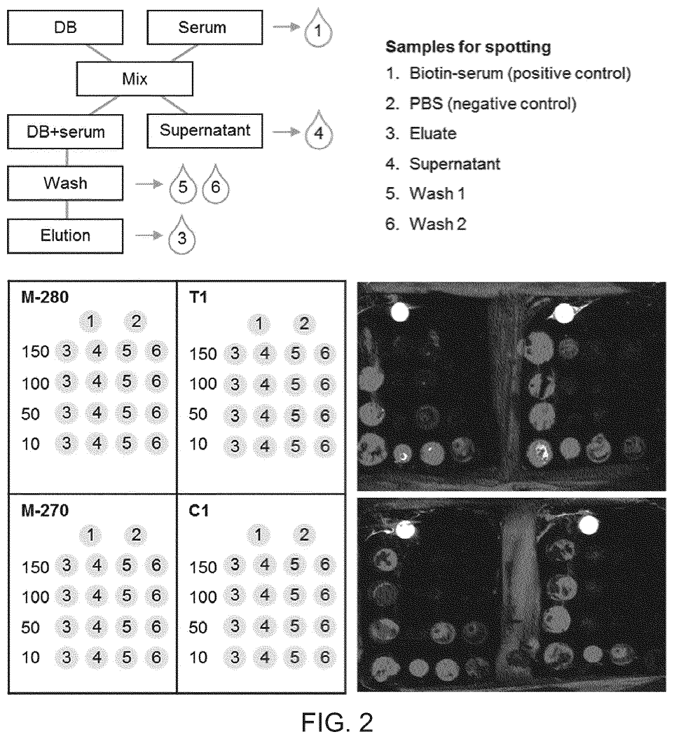

FIG. 2. Four types of Streptavidin coated Dynabeads™ (M-280, MyOne T1, M-270, MyOne C1) were evaluated in terms of binding capacity to biotinylated proteins in a serum sample. Serum was mixed with the different bead types washed and bound proteins were eluted. One microliter representing eluted proteins (3), supernatant (4) wash fractions (5, 6) were spotted onto Maxisorp slides and any present proteins were detected using Streptavidin-Alexa 647 fluorophore. Biotinylated serum proteins were used as positive control (1) and PBS as negative control (2). Similar binding capacities were observed for all bead types.

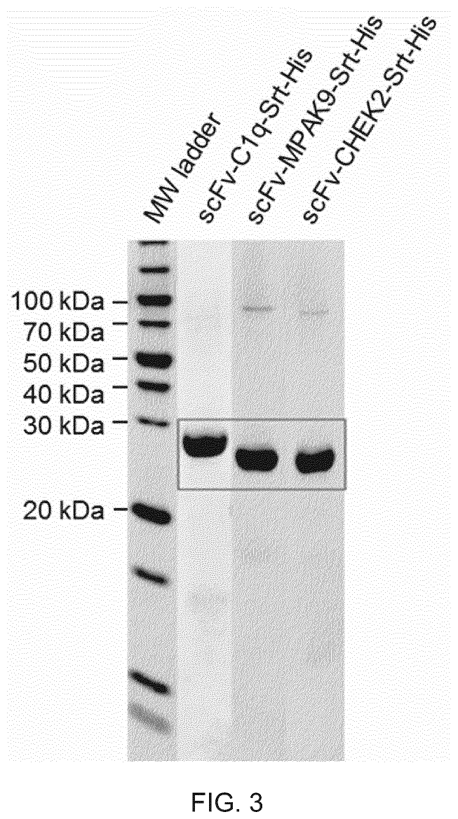

FIG. 3. The SDS-PAGE shows eluted fractions after IMAC purification of the protein scFv-C1Q-Srt (29.65 kDa), scFv-2-Srt (27.58 kDa) and scFv-3-Srt (27.69 kDa). From right to left: Protein ladder, Elution fraction of scFv-C1Q-Srt, Elution fraction of scFv-1-Srt and scFv-3-Srt.

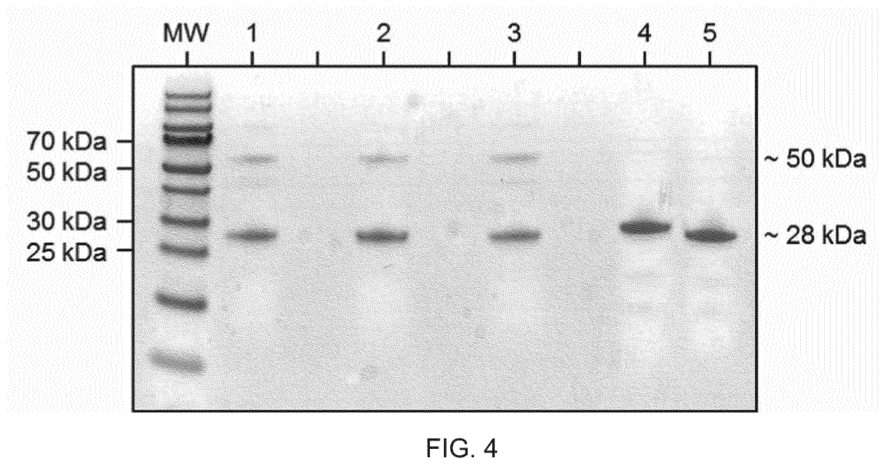

FIG. 4. SDS-PAGE shows oligonucleotide conjugated scFv-His₆(C1q) antibodies (50 kDa) in lane 1, 2 and 3. Bands corresponding to 28 kDa represent unconjugated scFv-His₆(C1q) antibodies (lane 1, 2, 3). Lane 4 and 5 contain unconjugated scFv-His₆(C1q) antibodies, included as controls.

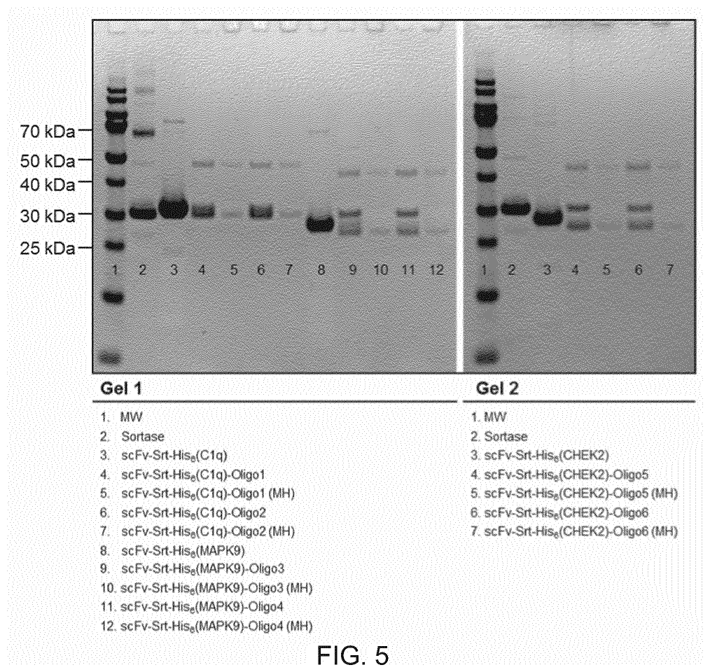

FIG. 5. Reducing SDS-PAGE showing oligonucleotide conjugated scFv-Srt-His₆ antibodies (C1q, MAPK9 or CHEK2). Gel 1 include protein ladder (MW), Sortase A enzyme (lane 2) conjugated antibodies scFv-Srt-His₆(C1q) or scFv-Srt-His₆(MAPK9) (lanes 4, 6, 9 and 11) purified conjugates using MagneHis beads (MH) (lanes 5, 7, 10 and 12). Gel 2 include protein ladder (lane 1), Sortase A enzyme (lane 2), unconjugated scFv-Srt-His₆(CHEK2) (lane 3), conjugated scFv-Srt-His₆(CHEK2) (lane 4 and 6) and purified conjugates (lane 5 and 7). Conjugates correspond to a protein band of approximately 50 KDa, unconjugated scFv to approximately 28 kDa and Sortase enzyme to 30 kDa.

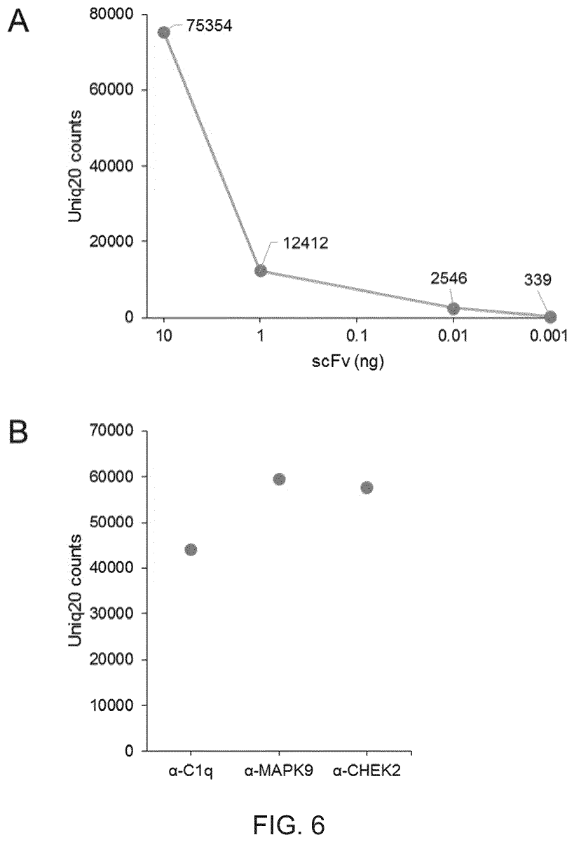

FIG. 6. Sequence count results generated from NGS using titrated amounts of a barcoded scFv-His₆(C1q) antibody (A) and three barcoded scFv-Srt-His₆ antibodies targeting C1q, MAPK9 and CHEK2 (B).

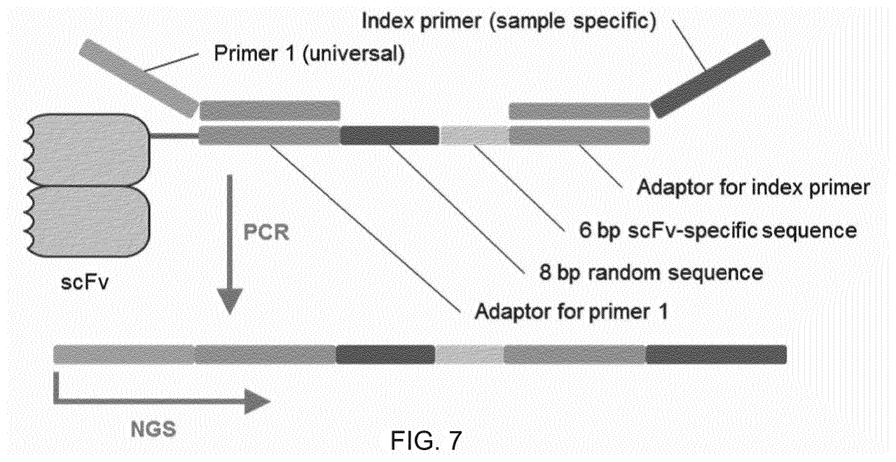

FIG. 7 (=Figure S1). The oligonucleotide sequences (66 bp) were designed to include an 8 bp long random sequence which were directly followed by a 6 bp scFv-specific barcode sequence. The barcode sequence represents the unique protein identifier whereas the random sequence allows filtering of only unique sequence counts.

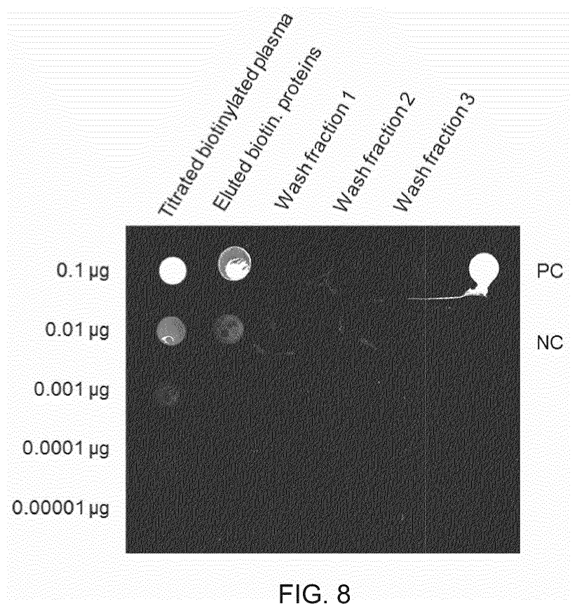

FIG. 8 (=Figure S2). Evaluation of biotinylated plasma proteins captured to beads.

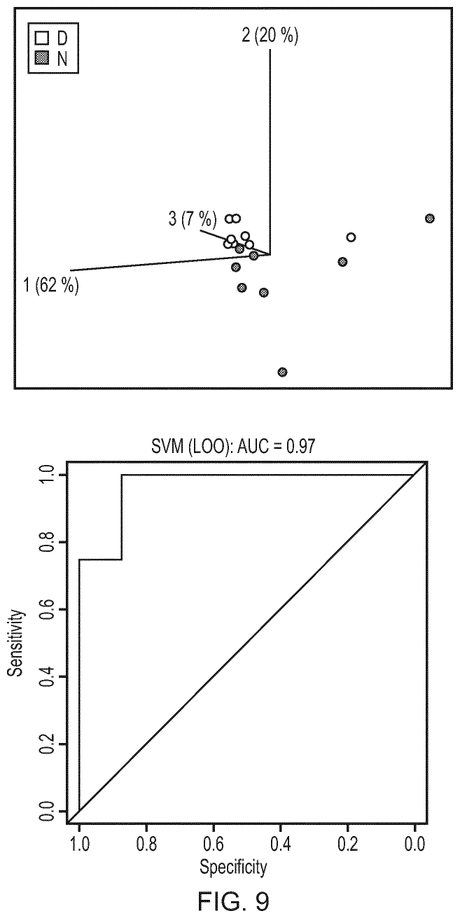

FIG. 9. Principle Component Analysis (PCA) plot representing 8 healthy (dark shading) and 8 PDAC samples (light shading) and ROC curve from SVM LOO CV analysis.

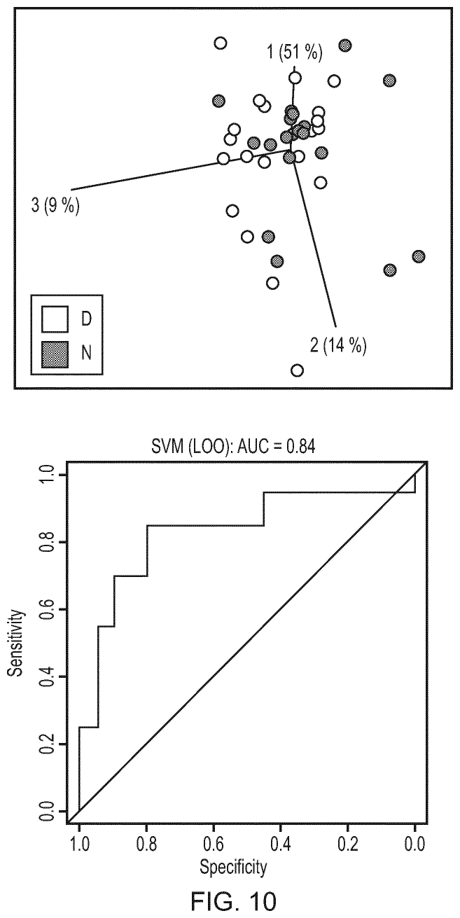

FIG. 10. Principle Component Analysis (PCA) plot representing 20 healthy (dark shading) and 20 PDAC samples (light shading) and ROC curve from SVM LOO CV analysis.

### EXAMPLES

Abstract

The search for biomarkers for improved clinical decision making and therapy constitute a major focus for technological research development within the field of proteomics. Affinity proteomics utilizing antibodies constitute a powerful and sensitive technique for multiplex protein expression profiling of complex samples, such as serum. However, the technique is associated with logistical and technical challenges and a biomarker discovery tool, capable to perform multiplex, sensitive and quantitative protein expression profiling in a high through put manner, is still lacking. This section describes an exemplified embodiment of the invention, referred to as Multiplexed Immuno-Assays in Solution (MIAS), which has been specifically developed to meet these key technical challenges. Specifically, engineered recombinant single-chain fragment variable (scFv) antibodies are site-specifically conjugate with unique DNA barcode sequences, as exemplified in a 1:1 manner, using Sortase mediated coupling strategy. These barcoded antibodies are then mixed with biotinylated serum proteins, already coupled to streptavidin coated magnetic beads, and bound antibodies are detected using next generation sequencing for a multiplex, sensitive and quantitative read-out. Proof-of-concept of the individual steps as well as of the principle of the MIAS platform were generated by using three recombinant scFv antibodies, each specifically targeting different proteins in a crude serum sample, and a NGS-based detection read-out. This demonstrates that MIAS is a highly useful novel tool for biomarker (in particular protein expression) profiling. Blood-based biomarkers will play a major role for future disease proteomics, where novel tools such a MIAS, could provide for a multiplex, ultra-sensitive and quantitative read-out.

1. Introduction

Detailed study of changes in the serum proteome between normal and diseased states holds promise for future biomarker identification and improved personalized medicine (Borrebaeck 2017). Antibody-based microarrays have during the last decade rapidly emerged as a unique and highly sensitive tool for multiplex protein expression profiling in complex samples (Haab et al 2001, Schroder et al 2013, Sjoberg et al 2016, Wingren and Borrebaeck 2009). The structure and format of the technology allows for a versatile probe choice in terms of polyclonal or monoclonal antibodies (Nilsson et al 2005, Stoevesandt and Taussig 2012), DARPins (Binz et al 2003) or affibodies (Nord et al 1997). The antibody design, however, play a crucial role for optimal array performance as the majority of off-the shelf readily polyclonal and monoclonal antibodies display impaired on-chip performance (Haab et al 2001, MacBeath 2002). By using phage display selected recombinant antibodies these provide a renewable source of antibodies, particularly suited for microarray (Wingren and Borrebaeck 2009). Antibody microarray platforms specifically developed for protein expression profiling using only minute amounts of serum sample have so far successfully delivered candidate biomarker signatures in both cancer (Carlsson et al 2008, Wingren et al 2012) and autoimmunity (Carlsson et al 2011, Delfani et al 2017). Despite the success, the antibody microarray technology has in its current use and formats some technical limitations which restrict their final implementation as truly large-scale biomarker discovery platforms. For example, each antibody may need to be individually produced, purified and dispensed via absorption onto the array creating logistical problems and inflicting overall array applicability. Also, with increasing demand for up-scaled multiplexity, miniaturized arrays in nanoscale format, are technically more challenging to produce and smaller spot size are more vulnerable from surrounding particles like dust (Petersson et al 2014a, Petersson et al 2014b, Petersson et al 2014c). The above listed key issues can be bypassed by using solution-based arrays thereby circumventing the need for planar microarrays and their associated technical limitations. Solution-based bead arrays interfaced with flow cytometry detection systems already exist and have been demonstrated to constitute a tool for protein profiling (de Jager et al 2003, Hodge et al 2004). However, restriction in multiplexity (<100 targets/assay) and the issue of generating sandwich pair antibodies create a logistical challenge when aiming for a global protein profiling tool (de Jager and Rijkers 2006, Elshal and McCoy 2006, Schwenk et al 2008). Another key issue related to current antibody microarray technologies is the use of fluorescence as detection signal as it only provides relative protein levels, thereby generating fold-changes in expression values and not absolute expression values. Also, sensitivity may be an issue as there is a risk of missing low abundant proteins. This can be addressed by using antibodies labelled with unique DNA barcode sequences thereby enabling the use of novel ultrasensitive, quantitative detection strategies, such as next-generation sequencing (NGS) and very suitable for solution-based bead array. The concept of using barcoded antibodies for protein expression profiling is not new and are currently being used in different immunoassays such as Proximity Ligation Assay (PLA), Proximity Extension Assay (PEA) (Darmanis et al 2011, Fredriksson et al 2002, Lundberg et al 2011). However, these methods rely on non-specific covalent oligonucleotide-tagging (targeting available primary amines), which result in impaired antibody function and deliver a heterogeneous mix of conjugated antibodies. This issue can be avoided by using specifically designed recombinant antibodies to be site-specifically conjugated with oligonucleotides as described herein, and as exemplified in a 1:1 manner.

The current invention addresses all above issues by developing Multiplex Immuno-Assay in Solution (MIAS), a novel tool for highly sensitive, quantitative and multiplex protein profiling. The exemplified assay utilizes specifically engineered recombinant single-chain fragment variable (scFv) antibodies that are site-specifically conjugated to oligonucleotide barcodes, in a 1:1 manner, using Sortase mediated coupling strategy. The barcoded antibodies are then mixed with biotinylated serum proteins, already coupled to streptavidin coated magnetic beads, and bound antibodies are detected using next generation sequencing for a multiplex, sensitive and quantitative read-out (FIG. 1).

In this section, the basic steps of an exemplary embodiment of the methods of the invention are present, in which 1) proteins present in a biotinylated serum sample are captured on magnetic beads, 2) the bead-coupled proteins are contacted with oligonucleotide-conjugated antibodies targeting up to three different proteins (CHEK2, MAPK9 and C1q) and 3) the antibodies bound to the proteins from the serum sample are successfully detected using NGS.

2. Materials and Methods

2.1 Sample Preparation and Capture to Beads

A limited set of de-identified human serum and plasma samples were collected at Skåne University hospital (Lund, Sweden). It should be noted that no clinical information or patient identifiers were retained for the samples since this information was neither needed nor used in this study. After completion of the study, the samples were thrown away.

Four different types of streptavidin-coated magnetic Dynabeads (M-280, MyOne T1, M-270, MyOne C1) (Life Technologies) were used to evaluate protein binding of all proteins present in biotinylated serum and plasma samples. Non-fractionated serum or plasma samples, previously biotinylated using EZ-Link Sulfo-NHS-LC-Biotin (Pierce, Rockford, Ill., USA) according to previously described protocols were used (Gerdtsson et al 2016, Ingvarsson et al 2007). Different volumes of biotinylated serum or plasma samples (representing ˜4-0.0002 μg of serum or plasma samples) were mixed with 500, 250, 150, 100, 50 or 10 μl of beads (see Results for combinations of beads and volumes) and incubated for 30 minutes in room temperature using gently agitation, according to the manufacturer's recommendation. The tubes, now containing a mixture of either serum or plasma and Dynabeads (DB), were placed in a magnetic holder and the supernatant, containing unbound proteins, was transferred to a new tube and labeled. DB were washed three times with 500 μl of washing buffer (PBS+0.05% (v/v) Tween-20) with transferal of the supernatants to new tubes after each round of washing. The DB/sample mixture were boiled in 0.1% (v/w) SDS at 95° C. for 10 minutes, to release bound proteins. Each fraction (0.5-1 μl) was manually spotted on a Black Polymer Maxisorp slide (NUNC A/S, Roskilde, Denmark) and allowed to dry. As negative and positive controls, PBS and serial dilutions of starting sample, respectively, were used. A DAKO hydrophobic pen (Thermo Fisher) was used to create a “reaction well” around the spotted area. The secured area, i.e. the array, was blocked with 100 μl of PBS (1% (w/v) milk and 1% (v/v) Tween-20) and incubated in a humidity chamber for 1 hour. Next, the slide was washed by adding 100 μl of washing buffer to each corner (4 corners in total) of the well and the procedure was repeated twice 100 μl of Alexa Fluor 647-conjugated streptavidin (SA-647, 1 μg/ml), diluted in blocking buffer, was added and incubated for 1 hour followed by three rounds of washing as previously described. The slide was quickly rinsed in MQ water and dried using N₂-gas. Slides were scanned in a microarray scanner (ScanArray Express, Perkin Elmer Life & Analytical Sciences) at 10 μm resolution and spot morphology and signal intensities were inspected.

2.2 Production of ScFv-His₆(C1Q) Antibodies

The α-C1q recombinant scFv antibody (scFv-His₆(C1q)) was selected from in-house designed large phage-display library (Soderlind et al 2000) and produced as previously described (Ingvarsson et al 2007). In brief, 0/N cultures of E. coli were grown with appropriate antibiotics at 37° C. and induced with 1 mM isopropyl β-D-1-thiogalactopyranoside (IPTG) when OD reached 0.9-1.0. Antibodies were purified using MagneHis purification system according to manufacturer's recommendation followed by buffer exchange to PBS using Zeba 96-well desalt spin plates (Pierce). Purity and concentration was evaluated using 10% SDS-PAGE (Invitrogen, Carlsbad, Calif., USA) and a Nanodrop-1000 spectrophotometer at 280 nm (Thermo Scientific, Wilmington, Del., USA).

2.3 Generation and Production of ScFv-LPETG Antibodies

Three single-chain variable fragments (scFv), targeting C1q, MAPK9 and CHEK2, selected from in-house designed large phage-display libraries (Sall et al 2016, Soderlind et al 2000), were each used as template in a PCR reaction with primers introducing an N-terminal NcoI restriction endonuclease site, and a C-terminal (GS)₃—Srt-XhoI (Srt=LPETG, Sortase tag) sequence. The generated PCR products were further used for insertion into a pET-26b(+) vector (Novagen), harbouring an N-teminal pelB signal sequence and a C-terminal His₆ tag, generating the three scFv gene constructs pelB-scFv-(GS)₃-Srt-His₆. The final gene constructs pelB-scFv(C1q)-(GS)₃-Srt-His₆, pelB-scFv(MAPK9)-(GS)₃-Srt-His₆ and pelB-scFv(CHEK2)-(GS)₃-Srt-His₆ were sequence verified by DNA sequencing.

All three scFv-Srt-His₆ constructs were transformed into E. coli BL21(DE3) cells (Merck Biosciences). For each construct, a small (20 mL) start culture in TSB/Y medium (30 g tryptic soy broth, 5 g yeast extract, 1 L deionized water), supplemented with 1% (v/w) glucose and 25 μg/mL kanamycin, was prepared, followed by overnight incubation at 37° C. (with shaking). A larger bacterial culture was prepared for each construct from 500 mL of TSB/Y medium, supplemented with 0.125 M sucrose, 25 μg/mL kanamycin and 10 mL of inoculum. The cultures were incubated at 37° C. (with shaking) and allowed to reach an OD₆₀₀ value of 0.6-1.0. Protein expression was induced by addition of 1 mM IPTG, followed by incubation at 30° C. for 2 h. The cells were harvested by centrifugation for 15 min (4648 rcf, +4° C.), after which cell lysis was performed by resuspending the pellet in 40 mL of IMAC binding buffer (20 mM phosphate buffer, 500 mM NaCl, 40 mM imidazole, pH 7.4), supplemented with 800 μL lysozyme (50 mg/mL), 10 μL DNase I and 25 μL MgCl₂ (1 M), prior to incubation for 30 min at room temperature. Lysed cells were centrifuged (30 min, 21612 rcf, +4° C.) and the resulting supernatant was further used for protein purification. Purification was performed using immobilized metal ion affinity chromatography (IMAC) on an ÄKTAxpress system (GE Healthcare) with Chelating Sepharose™ Fast Flow matrix (GE Healthcare) loaded with Zn²⁺ ions. The column was first equilibrated with IMAC binding buffer, prior to loading of the cell lysate and subsequent washing with binding buffer. Protein elution was performed with 20 mM sodium phosphate, 500 mM NaCl, 500 mM imidazole (pH 7.4), followed by dialysis (6-8,000 MWCO) against 50 mM Tris buffer (pH 7.5) overnight at +4° C. The proteins were then stored at +4° C. until further use. Protein absorbance at 280 nm was used to estimate the concentration of each scFv-Srt-His6 protein. For eluted protein fractions, samples were taken for SDS-PAGE analysis, which was performed on a 12% gel under reducing conditions.

2.4 Design of Oligonucleotide Sequences

The oligonucleotide sequences (66 bp) were designed to include an 8 bp long random sequence (position 27-35) which were directly followed by a 6 bp specific barcode sequence (position 36-40) (Figure S1). The sequences of all oligonucleotide sequences are summarized in Supplemental Table 1. The barcode sequence represents the unique protein identifier whereas the random sequence allow calculation of only unique sequence counts. The oligonucleotides were designed to carry either a thiol or a tri-glycine modification in the 5′end. Thiol modified oligonucleotides were purchased from Sigma and tri-glycine modified oligonucleotides from Biomers (Ulm, Germany).

2.5 Thiol-Maleimide Conjugation of ScFv-His6 and Oligonucleotides

Conjugation of the recombinant C1q scFv antibody (scFv-His₆(C1q)) to thiol-modified oligonucleotides was performed by mixing 20 μg (2 μg/μl) of scFv-His₆(C1q) with 1 μl of 4 mM sulfo-SMCC and allowed to incubate at room temperature for 2 h (Nong et al 2013). Three microliters of 100 μM oligo was reduced with 12 μl of 100 mM DTT and incubated for 1 hour at 37° C. Prior to conjugation, the antibody and the oligos were separately exchanged to conjugation buffer (PBS, 0.1 M EDTA) using Microspin G50 columns according to the manufacturer's recommendation. The activated antibody and oligo was mixed and dialyzed 0/N against 1×PBS using 3.5 kD dialysis cups. Conjugation was verified using non-reducing 10% SDS-PAGE and Commassie staining. Efficacy of the conjugation was estimated using Qubit ssDNA Assay kit (Thermo Scientific, Wilmington, Del., USA) and Nanodrop.

2.6 Sortase-Mediated Conjugation of ScFv-Srt-His6 Antibodies and Oligonucleotides

Oligonucleotides, carrying a tri-glycine (G-G-G) in their 5′-end, were used for site-specific, enzyme dependent conjugation to scFv-Srt-His₆ antibodies. In short, purified scFv-Srt-His₆ antibodies were buffer exchanged to 500 μl of sortase ligation buffer (50 mM Tris, 150 mM NaCl, 10 mM CaCl₂, pH 7.5) and concentrated using Amicon Ultra 10K 0.5 ml centrifugal filters (Westerlund et al 2015). Recombinant scFv-Srt-His₆ antibodies were mixed with oligonucleotides in a 1:1 ratio (0.1 nmol) and 5 μl of 10 μM high-activity mutant Sortase A (kindly provided by M. Hedhammar) in ligation buffer (50 μl total reaction volume). The conjugation mixtures were placed 0/N on a shaker at room temperature. scFv-Srt-His₆G-oligo solution (45 μl) was mixed with 30 μl Magne-His (Promega) beads and incubated for 10 min to remove His-tagged content (non-conjugated scFv-Srt-His₆, byproduct and Sortase). The bead mix was place on magnet for 2 min and the supernatant containing purified scFv-Srt-His₆-oligo was extracted.

2.7 MIAS Using Barcoded ScFv

Proof-of-concept for the MIAS assay was generated in two different set-ups, using 1) thiol-maleimide coupled scFv-oligos and 2) sortase conjugated scFv-LPETG-oligos. In the first set-up, 1 μl (2 mg/ml) of biotinylated serum sample was mixed with 500 μl Dynabeads® M-280 (Life Technologies) in four separate tubes and incubated and washed as described in 2.1. Next, serial dilutions (10, 1, 0.01 and 0.001 ng respectively) of α-C1q-oligo (thiol-maleimide conjugated) were added to separate tubes and incubated for 2 h at 4° C. with gentle agitation. Unbound α-C1q-oligo were removed by washing three times with 500 μl of wash buffer (PBS+0.05% Tween-20). After the last wash, the beads were resuspended in 50 μl of nuclease free water and used for PCR and NGS.

In the second set-up, three different scFv-Srt-His₆-oligos targeting the serum proteins C1 q, MAPKK9 and CHEK2, were used. In brief, 1 ul of serum (2 mg/ml) were incubated with 75 μl of streptavidin coated Dynabeads® M-270 (DB) (Life Technologies). The beads were then washed as described above and 6 μl of each purified scFv-Srt-His₆-oligo was added. After 2 h of incubation and washing, the beads were resuspended in 50 μl of nuclease-free water and used for PCR and NGS.

2.8 Library Preparation and Next Generation Sequencing

For adapter PCR, 8 μl of each bead mix (˜5 ng DNA) was mixed with 1× Phusion Master Mix (Thermo Scientific #F-531), 0.5 μM Illumina adapter, index primer (corresponding to each sample) and nuclease free water in a total volume of 20 μl. PCR program: 98° C. 2 min; 25 repeats of: 98° C. 20 s, 65° C. 30 s, 72° C. 30 s; 72° C. 5 min; 10° C. PCR purification was performed using Agencourt® AMPure XP beads according to manufacturer's recommendation (1.8 ratio). Positive controls contained pure oligos (no scFv) and negative control (water). Quality control of purified PCR products was done using Bioanalyzer and Agilent High Sensitivity DNA kit. 10 μl of each sample was pooled and diluted to a total concentration of 2 nM before sequencing on a HiSeq 2500 (Illumina) (first set-up) and MiSeq (second set-up).

2.9 NGS Data Analysis

Based on the index sequence NGS data was de-multiplexed for further analysis. As a quality check, possible contaminated and/or mismatched sequences were excluded by filtering for sequences that contained the correct 5′-adaptor sequence (AGATCGGAAGAGCACACGTCTGAACT). In a second step, sequences were matched according to barcode specific sequences (including random sequence) allowing up to one mismatch (Phred Quality score of Q20) (Ewing and Green 1998, Ewing et al 1998). Thereafter, only unique sequences for each barcode were filtrated and counted, generating a final sequence count (UniQ20) for each scFv. Each specific scFv were represented by two barcode sequences (separately labelled and pooled before assay) were used to avoid biased detection read out in the NGS.

3. Results

In this technical study, we have generated the first proof-of-concept for the MIAS set-up for sensitive detection of proteins in serum (FIG. 1). The method utilized specifically 1:1 DNA-labelled recombinant scFv-Srt-His₆ antibodies (denoted barcoded scFvs) in an in-solution bead-based antigen binding set-up. Bound antibodies are than detected using NGS as a read-out.

3.1 Sample Capture on Beads

As a first step, we investigated if all proteins present in a complex serum or plasma sample could be captured to magnetic beads. For this purpose, titrated amounts of biotinylated serum/plasma samples were mixed with Streptavidin coated magnetic beads. The binding capacity of four types of magnetic beads (Dynabeads: M-280, MyOne T1, M-270, and MyOne C1) were evaluated in terms of bead volume, sample volume and sample type (serum, plasma). Sampling were made during the loading and washing process and manually spotted on slides and visualized using streptavidin-Alexa-647 (FIG. 2). Indeed, all four bead types demonstrated high binding capacities, with the ability to capture all proteins that were present in the samples, irrespectively if it was a serum or plasma sample (Figure S2). No major differences were observed between the different bead types. Based from the titration experiments, the results showed that when using bead volumes of less than 100 μl, the risk of saturation/over loading of the beads (when using ˜2 μg of sample) was increased. This referred to all bead types. From these experiments, we concluded that all proteins present in a biotinylated complex sample could be captured onto beads when using a bead volume of at least 100 μl (and 2 μg sample) and the choice of bead type was flexible.

3.2 Generation of LPETG Engineered Recombinant ScFv Antibodies

The concept of the MIAS set-up highly depend on the use of barcoded antibodies that have been site-specifically 1:1 conjugated to oligonucleotides. For this purpose, three recombinant scFv antibodies were genetically engineered to harbour the Sortase A recognition motif LPETG, which allows for specific conjugation to a glycine modified molecule, in this case, the oligonucleotide. All three constructs (scFv-Srt-His₆(C1q), scFv-Srt-His₆(MAPK9) and scFv-Srt-His₆(CHEK2)) were sequence verified using sequencing and successfully produced and purified with concentrations ranging from 0.05-0.1 mg/ml. Protein bands corresponding to molecular weights around 29 kDa for scFv-Srt-His₆(C1 q) and 27 kDa for scFv-Srt-His₆(MAPK9) and scFv-Srt-His₆(CHEK2) verified that all antibodies carried the Sortase recognition tag (FIG. 3). In order to investigate whether the modification process had inflicted the overall antigen binding capacity of the newly produced constructs the activity was estimated using Octet analysis. The results showed retained activity for all antibodies compared to wildtype which is essential for optimal performance in MIAS.

3.3 Thiol-Maleimide ScFv-Oligo Conjugation

To be able to test and take each individual steps in the MIAS assay as far as possible, before using the actually intended Srt-His₆-oligo antibodies, we used standard covalent coupling chemistry to conjugate oligonucleotides to a recombinant scFv-His₆(C1q) antibody. The conjugation was partly successful, indicating a coupling efficiency of around 30%. From the gel electrophoresis, protein bands representing both conjugated scFv-His₆(C1q)-oligo (˜50 kDa) and non-conjugated scFv-His₆(C1q) (˜28 kDa) were clearly seen (FIG. 4). Since this method allow for conjugation of several oligonucleotides to each antibody, it was a surprise to see that no protein bands of size >50 kDa appeared. This suggest that, even though the coupling rate was quite low, antibodies were apparently only conjugated to one oligonucleotide. Although further experiments are required to elucidate the precise reason for this, it might be due to the smaller size of the scFv antibodies (compared to a IgG) and thereby a lower number of available primary amines. Purification using Amicon Ultra filters however, failed to remove non-conjugated scFv-His₆(C1q).

3.4 Sortase-Mediated scFv-Srt-His6-Oligo Conjugation

As described, the intended MIAS set-up highly depends on the use of specifically engineered scFv antibodies for site-specific, 1:1 oligonucleotide conjugation. To meet this purpose, our antibodies were genetically equipped with a Sortase recognition motif (scFv-Srt-His₆)) to enable conjugation to an oligonucleotide carrying a tri-glycine modification in the 5′end. Three scFv-Srt-His₆ antibodies targeting C1q, MAPK9, CHEK2, were used to generate scFv-Srt-His6-oligo conjugates. In order to remove any unwanted His₆-tagged content (non-reacted scFv-Srt-His₆, byproducts and Sortase A enzyme) the solutions were mixed with Ni-NTA magnetic beads (MagneHis). However, as seen in FIG. 5, this purification was not entirely successful, as protein bands around 30 kDa, most likely representing non-conjugated scFv-Srt-His₆ antibodies, were still observed. However, no residues of excessive Sortase enzyme were present which implied that the enzyme was successfully removed using this method. Protein bands corresponding to ˜50 kDa were seen which confirmed successful conjugation for all three scFv-Srt-His₆, although they did not reach a 100% coupling efficiency (FIG. 5). Altogether, these results demonstrated the potential of using Sortase A mediated strategy for specifically conjugation of recombinant scFv antibodies with oligonucleotides in a 1:1 manner.

3.5 MIAS Using Barcoded ScFvs

Proof-of-concept of the MIAS set-up was generated in two different steps. In a first experiment, the aim was to verify the different steps included in the MIAS set-up. To this end, the scFv-His₆(C1q) antibody was used as a model clone. Herein, the scFv-His₆(C1q)-oligo (generated by traditional covalent conjugation approach and not Sortase) construct was titrated and mixed with magnetic beads (Dynabeads M280™) in order to explore the detection range. Any unbound scFv-His₆(C1q)-oligo were washed away and the remaining bead mixture (containing bound scFv-His₆(C1q)-oligo) were used for adaptor PCR ligation and prepared for sequencing on a HiSeq 2500 (IIlumina). After de-multiplexing, sequences were matched and filtered according to the 5′-adaptor sequence (AGATCGGAAGAGCACACGTCTGAACT), barcode sequence and unique random sequences (allowing a mismatch of up to 1%) which ultimately generated in total sequence count (UniQ20) for each antibody. The graph in FIG. 6A shows that the number of sequences decreased in relation to the concentrations of used scFv-His₆(C1q)-oligo, reaching its maximum around UniQ20 80 000 and the minimum at around UniQ20 of 340 (FIG. 6A). These results were exciting since they demonstrated the potential of NGS to also detect very low sequence counts, which could be the case when detecting for low abundant proteins present in complex biological samples.

In the second set-up we aimed to use scFv-Srt-His₆-oligo antibodies which, in contrary to the former set up (utilizing a scFv-His₆-oligo antibody), were specifically designed to meet the intended purpose of MIAS, i.e. conjugation in a 1:1 manner. Here, multiplexity was demonstrated by using three scFv-Srt-His₆-oligo antibodies, specifically targeting C1q, MAPK9 and CHEK2. To this end, biotinylated serum proteins were captured on streptavidin-coated beads and mixed with a pre-mixed pool consisting of the three different barcoded antibodies. The assay was performed, and data analyzed and handled as previously described (except for being sequenced on a MiSeq instead of HiSeq). The data clearly showed that all three scFv-Srt-His₆-oligo could be detected. The sequence counts (UniQ20) ranged from 44 000 (scFv-Srt-His₆(C1q)-oligo), 59 000 (scFv-Srt-His₆(MAPK9)-oligo) and 57 000 (scFv-Srt-His₆(CHEK2)-oligo) (FIG. 6B). These results clearly demonstrated that recombinant scFv antibodies, barcoded in a 1:1 site-specific manner, thus could be used in a multiplex bead-based assay for protein detection in a complex biological sample. Interfaced with NGS provide for a quantitative read-out, which holds great promise for further development of the MIAS assay towards a highly sensitive, quantitative and multiplex assay for protein expression profiling.

4. Discussion

Recombinant antibody microarrays has been established as a powerful tool for multiplex protein expression profiling of complex biological samples (Wingren and Borrebaeck 2008, Wingren and Borrebaeck 2009, Wingren et al 2012). However, planar microarrays are associated with some technical and logistical challenges affecting overall array performance, sensitivity and detection (Borrebaeck and Wingren 2009). One major bottleneck can be the laborious and the time-consuming step of individually having to dispense each antibody onto the solid support (via absorption) and together with increasing demands for multiplexity this limits its use as a truly high-throughput protein expression profiling tool. Additionally some recombinant scFv antibody, microarrays which are made by absorption to the solid phase may lead to impaired antibody activity (Borrebaeck and Wingren 2009). In addition, the fluorescence-based read-out only provide relative signals. The methods of the present invention have been developed in order to address those four technical issues associated with current planar antibody microarray technology. Firstly, a solution-based assay has several advantages over the planar antibody microarray platform. No high-precision printing is needed and the assay becomes easier to scale up without the physical limitations of a microarray slide and spots. Although the scFvs used in this study are built on a framework proven to withstand the handling with immobilization and drying on the microarray slides, this harsh treatment was here avoided by maintaining the scFvs in solution. Secondly, the assay steps are possible to automate which minimizes the manual labor and can lead to better reproducibility. Especially the tedious step to quantitate the signal intensities is circumvented, as the NGS provides direct digital read-out (and absolute quantification numbers), which is easier and faster to process and a great advantage compared to the relative quantification generated by the traditional fluorescent-based set-up. Thus, a sequencing based approach is a key attribute of the methods of the invention as it provides an ultra-sensitive, absolute quantitative detection compared to the relative fluorescent signals in the microarrays.

Great efforts have been made in the last decades to develop necessary protein discovery tools needed for the deciphering of relevant biomarkers within various diseases (Hanash et al 2008). Serum is an attractive sample format due to the wealth of potential biomarkers it carries and also because it is easily accessible and minimally invasive (Pitteri and Hanash 2007). However, serum contains a vast range of proteins, present in varying concentrations. Although the best performing arrays are within the pM range of sensitivity, the issue of detecting also low abundant proteins in complex biological samples is still a challenge (Anderson and Anderson 2002). The use of barcoded antibodies are currently used in immunoassays such as PLA and PEA (Fredriksson et al 2002, Lundberg et al 2011). However, both techniques rely on the generation of sandwich pair antibodies, which increase the risk for detection by non-cognate antibody pairs that follows with increased multiplexity. ProteinSeq utilize the advantages of NGS, and has thereby already been able to demonstrate the great potential of a digital read-out within biomarker analysis (Darmanis et al 2011). Another tentative disadvantage is that these methods utilize full-length antibodies which rely on a traditional covalent (thiol-maleimide) oligonucleotide tagging procedure, which increase the risk for ending up with antibodies with impaired function and different numbers of oligonucleotides to each antibody. The use of recombinant antibodies libraries, however, enables the customization needed for 1:1 conjugation of scFv antibody and oligos. Thus, control of both the number of oligos per scFv antibody and the site for conjugation is ensured. In the present embodiment of the methods of the invention, recombinant scFv antibodies specifically engineered with a Sortase A recognition-motif (LPETG) were successfully barcoded using tri-glycine modified oligonucleotide sequences. The already large library of high-affinity binders could be further expanded to cover virtually the whole serum proteome, which is a significant advantage for biomarker discovery studies. Furthermore, the recombinant antibody production is rapid and provides a renewable source of scFv antibodies that bypass the need for animal use or hybridoma cultivation (Frenzel et al 2013). NGS has repeatedly established itself as a state-of-the art read-out technology, and used in various set-ups, such as ProteinSeq for providing highly sensitive data for biomarker identification (Darmanis et al 2011). Also, NGS provide additional benefits such as a very high capacity of multiplexing and sample throughput, all powerful attributes to provide a solid base for large scale biomarker discovery.

Thus, it is demonstrated that the methods of the invention can be used to simultaneously detect three antibodies targeting specific serum proteins which was enabled by using a sequence based approach (FIG. 6B). This is very amenable for scaling up the assay multiplexity, and in line with other results (Darmanis et al 2011). The first experiment explored different concentrations of one used scFvs-His₆(C1q)-oligo. Here, it was possible to detect barcode specific α-C1q-scFv present in very high levels (almost reaching saturated levels) as well as in very low concentrations (˜320 seq counts), in the same experiment. This result was important since biological complex samples, such as serum, include proteins in a wide range of concentrations.

Depending on the (clinical) research question at hand, the flexibility of the methods of the invention makes them suitable for protein expression profiling on both a global scale as well as in a more focused analysis. The conceptual idea behind the methods of the invention also opens up for other assay designs. For an analysis with limited number of targets, the assay can be modified with sandwich antibody pairs for primary binding with antibodies on beads and secondary detection using scFv-oligos. Novel high-performing proteomic research tools, such as the platform presented here, will potentially play a major role for future disease proteomics, allowing biomarkers profiling at a high specificity and sensitivity. Such methods may enable improved understanding of underlying disease biology, disease diagnostics, prognostics, classification and therapy.

```
Supplemental Table 1.
Oligonucleotide sequences
Barcodes
1 TTCCCTACACGACGCTCTTCCGATCTNNNNNNN
    NTCACTGAGATCGGAAGAGCACACGTCTGAACT
2 TTCCCTACACGACGCTCTTCCGATCTNNNNNNN
    NCTTGACAGATCGGAAGAGCACACGTCTGAACT
3 TTCCCTACACGACGCTCTTCCGATCTNNNNNNN
    NACAGGTAGATCGGAAGAGCACACGTCTGAACT
4 TTCCCTACACGACGCTCTTCCGATCTNNNNNNN
    NGGTACAAGATCGGAAGAGCACACGTCTGAACT
5 TTCCCTACACGACGCTCTTCCGATCTNNNNNNN
    NGCGAATAGATCGGAAGAGCACACGTCTGAACT
6 TTCCCTACACGACGCTCTTCCGATCTNNNNNNN
    NCGCTTAAGATCGGAAGAGCACACGTCTGAACT
Index primers
1 CAAGCAGAAGACGGCATACGAGATGCTACCGTG
    ACTGGAGTTCAGACGTGTGCTCTTC
2 CAAGCAGAAGACGGCATACGAGATGCTCATGTG
    ACTGGAGTTCAGACGTGTGCTCTTC
3 CAAGCAGAAGACGGCATACGAGATAGGAATGTG
    ACTGGAGTTCAGACGTGTGCTCTTC
4 CAAGCAGAAGACGGCATACGAGATTAGTTGGTG
    ACTGGAGTTCAGACGTGTGCTCTTC
5 CAAGCAGAAGACGGCATACGAGATATCGTGGTG
    ACTGGAGTTCAGACGTGTGCTCTTC
6 CAAGCAGAAGACGGCATACGAGATCGATTAGTG
    ACTGGAGTTCAGACGTGTGCTCTTC
7 CAAGCAGAAGACGGCATACGAGATGAATGAGTG
    ACTGGAGTTCAGACGTGTGCTCTTC
8 CAAGCAGAAGACGGCATACGAGATTGCCGAGTG
    ACTGGAGTTCAGACGTGTGCTCTTC
```

### REFERENCES

Anderson N L, Anderson N G (2002). The human plasma proteome: history, character, and diagnostic prospects. Molecular & cellular proteomics: MCP 1: 845-867.
Binz H K, Stumpp M T, Forrer P, Amstutz P, Pluckthun A (2003). Designing repeat proteins: well-expressed, soluble and stable proteins from combinatorial libraries of consensus ankyrin repeat proteins. Journal of molecular biology 332: 489-503.
Borrebaeck C A, Wingren C (2009). Design of high-density antibody microarrays for disease proteomics: key technological issues. Journal of proteomics 72: 928-935.
Borrebaeck C A (2017). Precision diagnostics: moving towards protein biomarker signatures of clinical utility in cancer. Nature reviews Cancer 17: 199-204.
Carlsson A, Wingren C, Ingvarsson J, Ellmark P, Baldertorp B, Ferno M et al (2008). Serum proteome profiling of metastatic breast cancer using recombinant antibody microarrays. Eur J Cancer 44: 472-480.
Carlsson A, Wuttge D M, Ingvarsson J, Bengtsson A A, Sturfelt G, Borrebaeck C A K et al (2011). Serum Protein Profiling of Systemic Lupus Erythematosus and Systemic Sclerosis Using Recombinant Antibody Microarrays. Mol Cell Proteomics 10.
Darmanis S, Nong R Y, Vanelid J, Siegbahn A, Ericsson O, Fredriksson S et al (2011). ProteinSeq: high-performance proteomic analyses by proximity ligation and next generation sequencing. PloS one 6: e25583.
de Jager W, to Velthuis H, Prakken B J, Kuis W, Rijkers G T (2003). Simultaneous detection of 15 human cytokines in a single sample of stimulated peripheral blood mononuclear cells. Clinical and diagnostic laboratory immunology 10: 133-139.
de Jager W, Rijkers G T (2006). Solid-phase and bead-based cytokine immunoassay: a comparison. Methods 38: 294-303.
Delfani P, Sturfelt G, Gullstrand B, Carlsson A, Kassandra M, Borrebaeck C A et al (2017). Deciphering systemic lupus erythematosus-associated serum biomarkers reflecting apoptosis and disease activity. Lupus 26: 373-387.
Elshal M F, McCoy J P (2006). Multiplex bead array assays: performance evaluation and comparison of sensitivity to ELISA. Methods 38: 317-323.
Ewing B, Green P (1998). Base-calling of automated sequencer traces using phred. II. Error probabilities. Genome research 8: 186-194.
Ewing B, Hillier L, Wendl M C, Green P (1998). Base-calling of automated sequencer traces using phred. I. Accuracy assessment. Genome research 8: 175-185.
Fredriksson S, Gullberg M, Jarvius J, Olsson C, Pietras K, Gustafsdottir S M et al (2002). Protein detection using proximity-dependent DNA ligation assays. Nature biotechnology 20: 473-477.
Frenzel A, Hust M, Schirrmann T (2013). Expression of recombinant antibodies. Frontiers in immunology 4: 217.
Gerdtsson A S, Dexlin-Mellby L, Delfani P, Berglund E, Borrebaeck C A, Wingren C (2016). Evaluation of Solid Supports for Slide- and Well-Based Recombinant Antibody Microarrays. Microarrays (Basel) 5.
Guimaraes C P, Witte M D, Theile C S, Bozkurt G, Kundrat L, Blom A E et al (2013). Site-specific C-terminal and internal loop labeling of proteins using sortase-mediated reactions. Nature protocols 8: 1787-1799.
Haab B B, Dunham M J, Brown P O (2001). Protein microarrays for highly parallel detection and quantitation of specific proteins and antibodies in complex solutions. Genome biology 2: RESEARCH0004.
Hanash S M, Pitteri S J, Faca V M (2008). Mining the plasma proteome for cancer biomarkers. Nature 452: 571-579.
Hodge G, Hodge S, Haslam R, McPhee A, Sepulveda H, Morgan E et al (2004). Rapid simultaneous measurement of multiple cytokines using 100 microl sample volumes—association with neonatal sepsis. Clinical and experimental immunology 137: 402-407.
Ingvarsson J, Larsson A, Sjoholm A G, Truedsson L, Jansson B, Borrebaeck C A et al (2007). Design of recombinant antibody microarrays for serum protein profiling: targeting of complement proteins. Journal of proteome research 6: 3527-3536.
Kim C H, Axup J Y, Schultz P G (2013). Protein conjugation with genetically encoded unnatural amino acids. Current opinion in chemical biology 17: 412-419.
Lundberg M, Eriksson A, Tran B, Assarsson E, Fredriksson S (2011). Homogeneous antibody-based proximity extension assays provide sensitive and specific detection of low-abundant proteins in human blood. Nucleic acids research 39: e102.
MacBeath G (2002). Protein microarrays and proteomics. Nature genetics 32 Suppl: 526-532.
Nienberg C, Retterath A, Becher K S, Saenger T, Mootz H D, Jose J (2016). Site-Specific Labeling of Protein Kinase CK2: Combining Surface Display and Click Chemistry for Drug Discovery Applications. Pharmaceuticals (Basel) 9.
Nilsson P, Paavilainen L, Larsson K, Odling J, Sundberg M, Andersson A C et al (2005). Towards a human proteome atlas: high-throughput generation of mono-specific antibodies for tissue profiling. Proteomics 5: 4327-4337.
Nong R Y, Wu D, Yan J, Hammond M, Gu G J, Kamali-Moghaddam M et al (2013). Solid-phase proximity ligation assays for individual or parallel protein analyses with readout via real-time PCR or sequencing. Nature protocols 8: 1234-1248.
Nord K, Gunneriusson E, Ringdahl J, Stahl S, Uhlen M, Nygren P A (1997). Binding proteins selected from combinatorial libraries of an alpha-helical bacterial receptor domain. Nature biotechnology 15: 772-777.
Parthasarathy R, Subramanian S, Boder E T (2007). Sortase A as a novel molecular “stapler” for sequence-specific protein conjugation. Bioconjugate chemistry 18: 469-476.
Petersson L, Berthet Duroure N, Auger A, Dexlin-Mellby L, Borrebaeck C A, Ait Ikhlef A et al (2014a). Generation of miniaturized planar ecombinant antibody arrays using a microcantilever-based printer. Nanotechnology 25: 275104.
Petersson L, Coen M, Amro N A, Truedsson L, Borrebaeck C A, Wingren C (2014b). Miniaturization of multiplexed planar recombinant antibody arrays for serum protein profiling. Bioanalysis 6: 1175-1185.
Petersson L, Dexlin-Mellby L, Bengtsson A A, Sturfelt G, Borrebaeck C A, Wingren C (2014c). Multiplexing of miniaturized planar antibody arrays for serum protein profiling—a biomarker discovery in SLE nephritis. Lab on a chip 14: 1931-1942.
Pitteri S J, Hanash S M (2007). Proteomic approaches for cancer biomarker discovery in plasma. Expert review of proteomics 4: 589-590.
Sall A, Walle M, Wingren C, Muller S, Nyman T, Vala A et al (2016). Generation and analyses of human synthetic antibody libraries and their application for protein microarrays. Protein engineering, design & selection: PEDS 29: 427-437.
Schroder C, Srinivasan H, Sill M, Linseisen J, Fellenberg K, Becker N et al (2013). Plasma protein analysis of patients with different B-cell lymphomas using high-content antibody microarrays. Proteomics Clinical applications 7: 802-812.
Schwenk J M, Gry M, Rimini R, Uhlen M, Nilsson P (2008). Antibody suspension bead arrays within serum proteomics. Journal of proteome research 7: 3168-3179.
Sjoberg R, Mattsson C, Andersson E, Hellstrom C, Uhlen M, Schwenk J M et al (2016). Exploration of high-density protein microarrays for antibody validation and autoimmunity profiling. New biotechnology 33: 582-592.
Soderlind E, Strandberg L, Jirholt P, Kobayashi N, Alexeiva V, Aberg A M et al (2000). Recombining germline-derived CDR sequences for creating diverse single-framework antibody libraries. Nature biotechnology 18: 852-856.
Stoevesandt O, Taussig M J (2012). Affinity proteomics: the role of specific binding reagents in human proteome analysis. Expert review of proteomics 9: 401-414.
Wingren C, Borrebaeck C A (2008). Antibody microarray analysis of directly labelled complex proteomes. Current opinion in biotechnology 19: 55-61.
Wingren C, Borrebaeck C A (2009). Antibody-based microarrays. Methods Mol Biol 509: 57-84.
Wingren C, Sandstrom A, Segersvard R, Carlsson A, Andersson R, Lohr M et al (2012). Identification of serum biomarker signatures associated with pancreatic cancer. Cancer research 72: 2481-2490.

### Example A

To further confirm the utility of the method of the invention (also referred to herein as the ProMIS' method) as a multiplex, solution-based, protein analysis tool, the method described above was used together with scFv antibodies targeting specificities previously associated with pancreatic cancer in order to successfully distinguish patients with pancreatic cancer.

Results

In accordance with the Materials and Methods set out below, two experimental setups were performed, each using binding moiety-oligonucleotide conjugates based on 16 scFv antibodies targeting specificities previously associated with pancreatic cancer (Gerdtsson et al. 2015; Mellby et al. 2018; Wingren et al. 2012) and serum samples from both healthy individuals and patients diagnosed with pancreatic cancer (pancreatic ductal adenocarcinoma (PDAC)). Sequencing was run using the IIlumina platform and the NextSeq series. Obtained sequencing data was normalised using the NormalyzerDE tool (Willforss et al. 2018). Normalised data was further explored and analyzed using Qlucore and by leave-one-out cross validation and Support Vector Machine (SVM LOO CV).

The two experimental setups and results are as follows:

1. A total of 16 scFv antibodies (Table 2) and 8 healthy and 8 PDAC serum samples. Data showed that healthy and PDAC samples could be separated with an AUC of >0.90 (FIG. 9).

2. A total of 16 scFv antibodies (same as previous run) and 20 healthy and 20 PDAC serum samples. Data showed that healthy and PDAC samples could be separated with an AUC of around 0.84 (FIG. 10). The lower AUC value in this run may be related to an observed sequencing artefact, but the outcome still confirms the concept.

TABLE 2
List of analytes targeted the 16 scFv
antibodies that were used in the two
ProMIS setups. Number within brackets
represent individual clone number.

| | scFv antibodies |
|---|---|
| No. | Antibody specificity |
| 1 | Complement C5 (1) |
| 2 | Complement C5 (2) |
| 3 | Plasma protease C1 inhibitor (1) |
| 4 | Plasma protease C1 inhibitor (2) |
| 5 | Interleukin-4 (1) |
| 6 | Interleukin-4 (2) |
| 7 | HADH2 protein |
| 8 | MCP-1 |
| 9 | Lewis x |
| 10 | Sialyl Lewis x |
| 11 | Complement C1q |
| 12 | Protein kinase C zeta type |
| 13 | Aprataxin and PNK-like factor |
| 14 | Cyclin-dependent kinase 2 |
| 15 | Vascular endothelial growth factor |
| 16 | Properdin |

These new data from two independent setups therefore further confirm the utility of the method of the invention as a multiplex, solution-based, protein analysis tool.

Example A Materials and Methods

As an extension of previous results and to explore the potential of the methods of the invention, herein also referred to as “ProMIS”, two independent setups including serum samples from both healthy individuals and patients diagnosed with pancreatic cancer were performed. The specificities of the antibodies were chosen based on previous association with pancreatic cancer (Gerdtsson et al., 2015; Gerdtsson et al., 2016; Mellby et al., 2018; Wingren et al., 2012). The number of scFv was limited to a maximum of 20. The underlying reason for this was that twenty was considered to more manageable in this early stage of technology development. The experiments were performed, based on the same protocols as previous (see Material and Methods section on pages 36-40) with some minor changes related to design of the oligonucleotides, scFv antibody production and data analysis.

Samples

A total of 40 human serum samples collected from 20 healthy individuals and 20 individuals diagnosed with pancreatic cancer (PDAC), stage IV, were used. PDAC samples were collected at diagnosis before the patients had received any treatment. The blood was allowed to clot for at least 30 minutes and then centrifuged at 1,500×g for 10 minutes at 4° C. and stored at −80° C. until analysis. None of the controls developed pancreatic cancer during a 5-year follow up. It should be noted that no other clinical information or patient identifiers were retained for the samples since this information was neither needed nor used in these experiments. The samples had already been labelled with biotin according to already optimized protocols (Carlsson et al., 2010; Gerdtsson et al., 2015; Wingren, Ingvarsson, Dexlin, Szul, & Borrebaeck, 2007) as described by Mellby et al. (Mellby et al., 2018).

Antibody Generation and Production

Twenty single-chain variable fragment (scFv) antibodies were selected. A list including the specific targets for each antibody can be seen in Table 3. Generation of scFv-LPETG was performed as previously described in the Materials and methods section 2.3 on page 37-38 and subsequently transformed into E. coli BL21(DE3) cells (Merck Biosciences). scFv-LPETG antibodies were produced as previously described (Ingvarsson et al., 2007) with some minor modifications. In brief, 0/N cultures of E. coli were grown with appropriate antibiotics at 37° C. and induced with 1 mM isopropyl 3-D-1-thiogalactopyranoside (IPTG) when OD reached 0.9-1.0. Purification was performed using the His MultiTrap™ FF system (GE Healthcare Life Sciences) according to the manufacturers recommendation, followed by concentration and buffer exchange to Sortase Ligation Buffer using Amicon Ultra 10K 0.5 ml centrifugal filters. Due to some delays in the time schedule, only 16 out of 20 scFv-LPETG antibodies was prepared in time and subsequently used in ProMIS (Table 3).

TABLE 3
List of analytes targeted by scFv antibodies that in the
end were used in the two ProMIS setups (x). Number
within brackets represent individual clone number.
scFv antibodies

| | | Used in |
|---|---|---|
| No. | Antibody specificity | ProMIS (x) |
| 1 | Complement C5 (1) | X |
| 2 | Complement C5 (2) | X |
| 3 | Plasma protease C1 inhibitor (1) | X |
| 4 | Plasma protease C1 inhibitor (2) | X |
| 5 | Interleukin-4 (1) | X |
| 6 | Interleukin-4 (2) | X |
| 7 | HADH2 protein | X |
| 8 | MCP-1 | X |
| 9 | Lewis x | X |
| 10 | Sialyl Lewis x | X |
| 11 | Complement C1q | X |
| 12 | Protein kinase C zeta type | X |
| 13 | Aprataxin and PNK-like factor | X |
| 14 | Cyclin-dependent kinase 2 | X |
| 15 | Vascular endothelial growth factor | X |
| 16 | Properdin | X |
| 17 | Apolipoprotein A1 | |
| 18 | Serine/threonine-protein kinase | |
| | MARK1 | |
| 19 | Plasma protease C1 inhibitor (3) | |
| 20 | BIRC2 | |

Barcoding

Triglycine modified oligonucleotides were designed as previously (see Materials and methods section 2.4 above, page 38-39 and FIG. 7) except for that the scFv-specific tag was now extended with 2 bases, from original 6 to 8 bases, resulting in a 68 bp long oligonucleotide. Index primers were purchased from IDT technologies (Belgium). Full sequences are listed in Table 4. Barcoding was performed as already described in Materials and methods section 2.6 above, page 39, though non-conjugated scFv-LPETG antibodies and Sortase was removed by five rounds of washing with 450 μl PBS in Amicon Ultra 30K 0.5 ml centrifugal filters.

ProMIS Assay

1 ul of biotinylated serum (2 mg/ml) were incubated with 75 μl of streptavidin-coated Dynabeads® M-280 (Life Technologies) and incubated for 30 min at RT with gentle agitation. Next, the beads were washed four times with 100 μl of wash buffer (PBS+0.05% Tween-20). 100 μl of each barcoded scFv was pooled, from which 30 μl of pool was added to each sample. Unbound scFv antibodies were removed by washing three times with 500 μl of wash buffer. After the last wash, the beads were resuspended in 50 μl of nuclease free water and used for PCR and NGS. Library preparation and quality control were performed as described in Materials and methods section 2.8 above, page 40). DNA concentration was determined using the Qubit™ dsDNA HS Assay Kit (Thermo Fisher Scientific) according to the manufacturer's recommendations. Sequencing was performed at the Center for Translational Genomics, Lund University and Clinical Genomics Lund, SciLifeLab using the NextSeq 550 High-Output Kit v2.5, single-end, and with the addition of 15% of Phi control.

Two independent rounds of setups were performed in which the first setup included 16 samples (8 healthy and 8 PDAC) and the second setup a total of 40 samples (20 healthy and 20 PDAC).

Data Analysis

For Demultiplexing and adapter trimming of the raw data (BCL files) generated from the NextSeq500, bcl2fastq script from IIlumina Inc was used (Illumina, 2017). Subsequently, demultiplexed data was ran through an in-house pipeline in written in Java programming language to count total number of UMIs (Unique Molecular Index) and scFv-specific tags for each sample and an overall summary of counts for all samples. For our analysis only reads that were passed through Passing Filter (PF %) and the quality over Q30, were used. Initial analysis showed good correlation between UMI and total count, and therefore the total counts, instead of UMI were used in further downstream analysis

Data was then normalized (Median) using NormalyzerDE (Willforss, Chawade, & Levander, 2018). Log2-tranformed, normalized data was then further explored and analysed using Qlucore Omice Explorer 3.1 software (Qlucore AB, Lund, Sweden). A linear support vector machine (SVM) combined with a leave-one-out classification algorithm was used to evaluate the predictive performance of a model, based on the 16 antibodies. A receiver operating characteristic (ROC) curve was constructed and the area under the curve (AUC) was then calculated and used as a measure of the prediction performance of the classifier (methods as described in Mellby et al 2018).

Table 4
Oligonucleotide sequences

| | | Barcode oligo |
|---|---|---|
| | | sequences |
| # | scFv tag | (5′-3′) |
| 1 | TGGCCT | TTCCCTACACGACGCTCTT |
| | AT | CCGATCTNNNNNNNNTGGC |
| | | CTATAGATCGGAAGAGCAC |
| | | ACGTCTGAACT |
| 2 | ACAGTA | TTCCCTACACGACGCTCTT |
| | TC | CCGATCTNNNNNNNNACAG |
| | | TATCAGATCGGAAGAGCAC |
| | | ACGTCTGAACT |
| 3 | GTTAGG | TTCCCTACACGACGCTCTT |
| | CA | CCGATCTNNNNNNNNGTTA |
| | | GGCAAGATCGGAAGAGCAC |
| | | ACGTCTGAACT |
| 4 | CACTAC | TTCCCTACACGACGCTCTT |
| | GG | CCGATCTNNNNNNNNCACT |
| | | ACGGAGATCGGAAGAGCAC |
| | | ACGTCTGAACT |
| 5 | TGGCTA | TTCCCTACACGACGCTCTT |
| | GA | CCGATCTNNNNNNNNTGGC |
| | | TAGAAGATCGGAAGAGCAC |
| | | ACGTCTGAACT |
| 6 | ACAGC | TTCCCTACACGACGCTCTT |
| | GCT | CCGATCTNNNNNNNNACAG |
| | | CGCTAGATCGGAAGAGCAC |
| | | ACGTCTGAACT |
| 7 | GTTAAC | TTCCCTACACGACGCTCTT |
| | TG | CCGATCTNNNNNNNNGTTA |
| | | ACTGAGATCGGAAGAGCAC |
| | | ACGTCTGAACT |
| 8 | CACTGT | TTCCCTACACGACGCTCTT |
| | AC | CCGATCTNNNNNNNNCACT |
| | | GTACAGATCGGAAGAGCAC |
| | | ACGTCTGAACT |
| 9 | TGCTTA | TTCCCTACACGACGCT |
| | CG | CTTCCGATCTNNNNNN |
| | | NNTGCTTACGAGATCG |
| | | GAAGAGCACACGTCTG |
| | | AACT |
| 10 | ACTCAG | TTCCCTACACGACGCT |
| | TA | CTTCCGATCTNNNNNN |
| | | NNACTCAGTAAGATCG |
| | | GAAGAGCACACGTCTG |
| | | AACT |
| 11 | CAGACC | TTCCCTACACGACGCT |
| | GT | CTTCCGATCTNNNNNN |
| | | NNCAGACCGTAGATCG |
| | | GAAGAGCACACGTCTG |
| | | AACT |
| 12 | TGCGG | TTCCCTACACGACGCT |
| | AGA | CTTCCGATCTNNNNNN |
| | | NNTGCGGAGAAGATCG |
| | | GAAGAGCACACGTCTG |
| | | AACT |
| 13 | ACTACT | TTCCCTACACGACGCT |
| | CG | CTTCCGATCTNNNNNN |
| | | NNACTACTCGAGATCG |
| | | GAAGAGCACACGTCTG |
| | | AACT |
| 14 | GTATAG | TTCCCTACACGACGCT |
| | TC | CTTCCGATCTNNNNNN |
| | | NNGTATAGTCAGATCG |
| | | GAAGAGCACACGTCTG |
| | | AACT |
| 15 | CAGCTC | TTCCCTACACGACGCT |
| | AT | CTTCCGATCTNNNNNN |
| | | NNCAGCTCATAGATCG |
| | | GAAGAGCACACGTCTG |
| | | AACT |
| 16 | GTAACT | TTCCCTACACGACGCT |
| | CC | CTTCCGATCTNNNNNN |
| | | NNGTAACTCCAGATCG |
| | | GAAGAGCACACGTCTG |
| | | AACT |
| | | Index Primer |
| | | sequences |
| # | Index | (5′-3′) |
| 1 | TCAGTC | CAAGCAGAAGACGGC |
| | CA | ATACGAGATGGACTG |
| | | AGTGACTGGAGTTCA |
| | | GACGTGTGCTCTTC |
| 2 | CGTAAT | CAAGCAGAAGACGGC |
| | GG | ATACGAGACCATTAC |
| | | GGTGACTGGAGTTCA |
| | | GACGTGTGCTCTTC |
| 3 | GTGTCA | CAAGCAGAAGACGGC |
| | TC | ATACGAGAGATGACA |
| | | CGTGACTGGAGTTCA |
| | | GACGTGTGCTCTTC |
| 4 | AACCG | CAAGCAGAAGACGGC |
| | GAT | ATACGAGAATCCGGT |
| | | TGTGACTGGAGTTCA |
| | | GACGTGTGCTCTTC |
| 5 | TCACCT | CAAGCAGAAGACGGC |
| | TG | ATACGAGACAAGGTG |
| | | AGTGACTGGAGTTCA |
| | | GACGTGTGCTCTTC |
| 6 | CGTTGC | CAAGCAGAAGACGGC |
| | AC | ATACGAGAGTGCAAC |
| | | GGTGACTGGAGTTCA |
| | | GACGTGTGCTCTTC |
| 7 | GTGAAG | CAAGCAGAAGACGGC |
| | CT | ATACGAGAAGCTTCA |
| | | CGTGACTGGAGTTCA |
| | | GACGTGTGCTCTTC |
| 8 | AACGTA | CAAGCAGAAGACGGC |
| | GA | ATACGAGATCTACGT |
| | | TGTGACTGGAGTTCA |
| | | GACGTGTGCTCTTC |
| 9 | TATTCG | CAAGCAGAAGACGGC |
| | TC | ATACGAGAGACGAAT |
| | | AGTGACTGGAGTTCA |
| | | GACGTGTGCTCTTC |
| 10 | CTAGGA | CAAGCAGAAGACGGC |
| | GG | ATACGAGACCTCCTA |
| | | GGTGACTGGAGTTCA |
| | | GACGTGTGCTCTTC |
| 11 | ACGAAT | CAAGCAGAAGACGGC |
| | CT | ATACGAGAAGATTCG |
| | | TGTGACTGGAGTTCA |
| | | GACGTGTGCTCTTC |
| 12 | GGCCT | CAAGCAGAAGACGGC |
| | CAA | ATACGAGATTGAGGC |
| | | CGTGACTGGAGTTCA |
| | | GACGTGTGCTCTTC |
| 13 | TATACT | CAAGCAGAAGACGGC |
| | CG | ATACGAGACGAGTAT |
| | | AGTGACTGGAGTTCA |
| | | GACGTGTGCTCTTC |
| 14 | CTACGG | CAAGCAGAAGACGGC |
| | AC | ATACGAGAGTCCGTA |
| | | GGTGACTGGAGTTCA |
| | | GACGTGTGCTCTTC |
| 15 | ACGTTC | CAAGCAGAAGACGGC |
| | GT | ATACGAGAACGAACG |
| | | TGTGACTGGAGTTCA |
| | | GACGTGTGCTCTTC |
| 16 | GGCGA | CAAGCAGAAGACGGC |
| | ATA | ATACGAGATATTCGC |
| | | CGTGACTGGAGTTCA |
| | | GACGTGTGCTCTTC |
| 17 | TAGTTC | CAAGCAGAAGACGGC |
| | GA | ATACGAGATCGAACT |
| | | AGTGACTGGAGTTCA |
| | | GACGTGTGCTCTTC |
| 18 | CTCGAA | CAAGCAGAAGACGGC |
| | CG | ATACGAGACGTTCGA |
| | | GGTGACTGGAGTTCA |
| | | GACGTGTGCTCTTC |
| 19 | ACTAGG | CAAGCAGAAGACGGC |
| | TT | ATACGAGAAACCTAG |
| | | TGTGACTGGAGTTCA |
| | | GACGTGTGCTCTTC |
| 20 | GGACCT | CAAGCAGAAGACGGCAT |
| | AC | ACGAGAGTAGGTCCGTG |
| | | ACTGGAGTTCAGACGTG |
| | | TGCTCTTC |
| 21 | CCATCC | CAAGCAGAAGACGGCAT |
| | GT | ACGAGAACGGATGGGTG |
| | | ACTGGAGTTCAGACGTG |
| | | TGCTCTTC |
| 22 | GTGATT | CAAGCAGAAGACGGCAT |
| | CA | ACGAGATGAATCACGTG |
| | | ACTGGAGTTCAGACGTG |
| | | TGCTCTTC |
| 23 | AGCCA | CAAGCAGAAGACGGCAT |
| | GTG | ACGAGACACTGGCTGTG |
| | | ACTGGAGTTCAGACGTG |
| | | TGCTCTTC |
| 24 | TATGGA | CAAGCAGAAGACGGCAT |
| | AC | ACGAGAGTTCCATAGT |
| | | GACTGGAGTTCAGACG |
| | | TGTGCTCTTC |
| 25 | GCTGTT | CAAGCAGAAGACGGCAT |
| | AA | ACGAGATTAACAGCGTG |
| | | ACTGGAGTTCAGACG |
| | | TGTGCTCTTC |
| 26 | CGGAG | CAAGCAGAAGACGGCAT |
| | CTG | ACGAGACAGCTCCGGTG |
| | | ACTGGAGTTCAGACG |
| | | TGTGCTCTTC |
| 27 | TTACCA | CAAGCAGAAGACGGCAT |
| | GC | ACGAGAGCTGGTAAGTG |
| | | ACTGGAGTTCAGACG |
| | | TGTGCTCTTC |
| 28 | AACTAG | CAAGCAGAAGACGGCAT |
| | CT | ACGAGAAGCTAGTTGTG |
| | | ACTGGAGTTCAGACG |
| | | TGTGCTCTTC |
| 29 | GTAGC | CAAGCAGAAGACGGCAT |
| | GGC | ACGAGAGCCGCTACGTG |
| | | ACTGGAGTTCAGACG |
| | | TGTGCTCTTC |
| 30 | CAGATC | CAAGCAGAAGACGGCAT |
| | CT | ACGAGAAGGATCTGGTG |
| | | ACTGGAGTTCAGACG |
| | | TGTGCTCTTC |
| 31 | TCTTAA | CAAGCAGAAGACGGCAT |
| | TG | ACGAGACATTAAGAGTG |
| | | ACTGGAGTTCAGACG |
| | | TGTGCTCTTC |
| 32 | AGCCGT | CAAGCAGAAGACGGCAT |
| | AA | ACGAGATTACGGCTGTG |
| | | ACTGGAGTTCAGACG |
| | | TGTGCTCTTC |
| 33 | GTACAG | CAAGCAGAAGACGGCAT |
| | AT | ACGAGAATCTGTACGTG |
| | | ACTGGAGTTCAGACG |
| | | TGTGCTCTTC |
| 34 | CAGTGT | CAAGCAGAAGACGGCAT |
| | CA | ACGAGATGACACTGGTG |
| | | ACTGGAGTTCAGACG |
| | | TGTGCTCTTC |
| 35 | TCTACC | CAAGCAGAAGACGGCAT |
| | GG | ACGAGACCGGTAGAGTG |
| | | ACTGGAGTTCAGACG |
| | | TGTGCTCTTC |
| 36 | AGCGTA | CAAGCAGAAGACGGCAT |
| | TC | ACGAGAGATACGCTGTG |
| | | ACTGGAGTTCAGACG |
| | | TGTGCTCTTC |
| 37 | ATGGTC | CAAGCAGAAGACGGCAT |
| | CT | ACGAGAAGGACCATGTG |
| | | ACTGGAGTTCAGACG |
| | | TGTGCTCTTC |
| 38 | CCTTGT | CAAGCAGAAGACGGCAT |
| | AG | ACGAGACTACAAGGGTG |
| | | ACTGGAGTTCAGACG |
| | | TGTGCTCTTC |
| 39 | GGACAA | CAAGCAGAAGACGGCAT |
| | TC | ACGAGAGATTGTCCGTG |
| | | ACTGGAGTTCAGACG |
| | | TGTGCTCTTC |
| 40 | TACACG | CAAGCAGAAGACGGCAT |
| | GA | ACGAGATCCGTGTAGTG |
| | | ACTGGAGTTCAGACG |
| | | TGTGCTCTTC |

### EXAMPLE A REFERENCES

Carlsson, A., Persson, O., Ingvarsson, J., Widegren, B., Salford, L., Borrebaeck, C. A., & Wingren, C. (2010). Plasma proteome profiling reveals biomarker patterns associated with prognosis and therapy selection in glioblastoma multiforme patients. Proteomics Clin Appl, 4(6-7), 591-602. doi:10.1002/prca.200900173
Gerdtsson, A. S., Malats, N., Sall, A., Real, F. X., Porta, M., Skoog, P., . . . Borrebaeck, C. A. (2015). A Multicenter Trial Defining a Serum Protein Signature Associated with Pancreatic Ductal Adenocarcinoma. Int J Proteomics, 2015, 587250. doi: 10.1155/2015/587250
Gerdtsson, A. S., Wingren, C., Persson, H., Delfani, P., Nordstrom, M., Ren, H., . . . Hao, J. (2016). Plasma protein profiling in a stage defined pancreatic cancer cohort—Implications for early diagnosis. Mol Oncol, 10(8), 1305-1316. doi:10.1016/j.molonc.2016.07.001
Illumina, I. (2017). bcl2fastq2 Conversion v2.19. User guide. Retrieved from https://support.illumina.com/content/dam/illumina-support/documents/documentation/software_documentation/bcl2fastq/bcl2fastq2_guide_15051736_v2.pdf
Ingvarsson, J., Larsson, A., Sjoholm, A. G., Truedsson, L., Jansson, B., Borrebaeck, C. A., & Wingren, C. (2007). Design of recombinant antibody microarrays for serum protein profiling: targeting of complement proteins. J Proteome Res, 6(9), 3527-3536. doi:10.1021/pr070204f
Mellby, L. D., Nyberg, A. P., Johansen, J. S., Wingren, C., Nordestgaard, B. G., Bojesen, S. E., Borrebaeck, C. A. K. (2018). Serum Biomarker Signature-Based Liquid Biopsy for Diagnosis of Early-Stage Pancreatic Cancer. J Clin Oncol, 36(28), 2887-2894. doi:10.1200/JCO.2017.77.6658
Willforss, J., Chawade, A., & Levander, F. (2018). NormalyzerDE: Online tool for improved normalization of omics expression data and high-sensitivity differential expression analysis. J Proteome Res. doi:10.1021/acs.jproteome.8b00523
Wingren, C., Ingvarsson, J., Dexlin, L., Szul, D., & Borrebaeck, C. A. (2007). Design of recombinant antibody microarrays for complex proteome analysis: choice of sample labeling-tag and solid support. Proteomics, 7(17), 3055-3065. doi:10.1002/pmic.200700025
Wingren, C., Sandstrom, A., Segersvard, R., Carlsson, A., Andersson, R., Lohr, M., & Borrebaeck, C. A. (2012). Identification of serum biomarker signatures associated with pancreatic cancer. Cancer Res, 72(10), 2481-2490. doi:10.1158/0008-5472.CAN-11-2883

1-78. (canceled)

79. A method of detecting and/or quantifying one or more biomarker(s) in a biological sample, the method comprising the steps of:
(a) providing a biological serum or plasma sample to be tested;
(b) contacting biomarkers present in the biological sample with one or more binding moiety-oligonucleotide conjugate(s) to generate biomarker-conjugate complexes, each conjugate comprising (i) a polypeptide binding moiety having binding specificity for one of the one or more biomarkers and (ii) an oligonucleotide moiety comprising an identifier nucleotide sequence which is indicative of the biomarker specificity of the binding moiety, wherein the binding moiety is conjugated to the oligonucleotide moiety in a one to one ratio by sortase-mediated site-specific conjugation at a single connection position on the binding moiety; and
(c) determining the nucleotide sequences of the oligonucleotide moieties in the binding moiety-oligonucleotide conjugates within the biomarker-conjugate complexes generated in step (b),
wherein the nucleotide sequences identified in step (c) are indicative of the presence and/or amount of the one or more biomarker(s) of interest in the biological sample,
wherein the method comprises multiplex biomarker analysis in solution.

80. The method of claim 79, wherein the binding moiety is an antibody or antigen-binding fragment thereof.

81. The method of claim 79, wherein the oligonucleotide moiety further comprises a first adaptor sequence for hybridising to a universal PCR primer and a second adaptor sequence for hybridising to a sample-specific PCR primer.

82. The method of 79, wherein the biomarker(s) is/are selected from the group consisting of: a peptide, a protein, a carbohydrate, a nucleic acid, a lipid, and a small molecule.

83. The method of claim 79, further comprising step (a′), following step (a), of immobilising the one or more biomarkers present in the sample on a substrate, optionally wherein:
the one or more biomarkers are immobilised on the substrate using molecular linkages; and/or
step (a′) comprises immobilising biotinylated biomarkers on a streptavidin-coated or avidin-coated substrate; and/or
the substrate is selected from the group consisting of particles, beads, superparamagnetic polymer beads, planar surfaces, and array plates; and/or
the method further comprises step (a″), following step (a′), of separating from the biological sample the immobilised biomarkers bound to the substrate.

84. The method of claim 79, wherein step (b) is performed in solution under conditions which enable the binding moiety-oligonucleotide conjugates to bind specifically to the biomarker(s) to which they are targeted.

85. The method of claim 79, further comprising step (b′), following step (b), of removing unbound binding moiety-oligonucleotide conjugates.

86. The method of claim 79, wherein step (c) comprises determining the nucleotide sequences of the oligonucleotide moieties within the binding moiety-oligonucleotide conjugates by DNA sequencing and/or RNA sequencing, optionally wherein the DNA sequencing and/or RNA sequencing comprises next generation nucleic acid sequencing, optionally selected from the group consisting of: real time sequencing, single molecule real time sequencing, pyrosequencing, Solexa sequencing, sequencing by ligation, SOLiD sequencing, Ion Torrent semiconductor sequencing, high-throughput sequencing systems, DNA nanoball sequencing, Nanostring, Heliscope single molecule sequencing, and nanopore sequencing.

87. The method of claim 79, further comprising step (d) of analysing the nucleic acid sequences identified in step (c) to categorise the biological sample, optionally wherein step (d) further comprises removing contaminated and/or mismatched sequences; and/or
further comprising determining the presence and/or quantity of the biomarkers in one or more positive and/or negative control samples alongside the biomarkers from the biological sample.

88. A binding moiety-oligonucleotide conjugate, wherein the binding moiety-oligonucleotide conjugate comprises (i) a binding moiety having binding specificity for a biomarker and (ii) an oligonucleotide moiety comprising an identifier nucleotide sequence which is indicative of the binding specificity of the binding moiety;
wherein the conjugation of the oligonucleotide moiety to the binding moiety is in a one to one ratio by sortase-mediated conjugation and is site-specific at a connection position on the binding moiety;
wherein the binding moiety is an scFv and comprises a single connection position.

89. The conjugate of claim 88, wherein the scFv is a mammalian scFv or a chimeric scFv;
wherein the scFv is a monoclonal scFv; and/or
wherein the scFv is a recombinant scFv.

90. The conjugate of claim 88, further comprising one or more means for conjugating the binding moiety to the oligonucleotide moiety, at the connection position.

91. The conjugate of claim 88, wherein the connection position is one or more amino acids within the binding moiety;
wherein the connection position is at the N-terminus of the binding moiety;
wherein the connection position comprises a sortase tag;
wherein the connection position comprises the amino acid sequence LPXTG, wherein X can be any amino acid;
wherein the connection position comprises the amino acid sequence (GS)ₙLPXTGₘ, wherein n is an integer between 1 and 6 and m is an integer between 1 and 6; and/or
wherein the connection position comprises or consists of the amino acid sequence (GS)₃LPXTG₃.

92. The conjugate of claim 88, wherein the oligonucleotide moiety is selected from the group consisting of DNA, RNA, morpholino, peptide nucleic acid (PNA), locked nucleic acid (LNA), glycol nucleic acid (GNA), threose nucleic acid (TNA), and derivatives thereof;
wherein the oligonucleotide moiety comprises DNA;
wherein the oligonucleotide moiety is 10 or more nucleotides in length;
wherein the oligonucleotide moiety is 50 to 100 nucleotides in length;
wherein the oligonucleotide moiety further comprises a random nucleotide sequence, optionally 3 or more nucleotides in length or 5 to 12 nucleotides in length; and/or
wherein the oligonucleotide moiety further comprises one or more adaptor sequences for hybridising to PCR primers, optionally wherein the oligonucleotide moiety further comprises a first adaptor sequence for hybridising to a universal PCR primer and a second adaptor sequence for hybridising to a sample-specific PCR primer.

93. The conjugate of claim 88, wherein the identifier nucleotide sequence is 3 or more nucleotides in length or wherein the identifier nucleotide sequence is 4 to 15 nucleotides in length.

94. A system for detecting and/or quantifying one or more biomarker(s) in a biological sample, comprising one or more populations of binding moiety-oligonucleotide conjugates of claim 88;
wherein each population of binding moiety-oligonucleotide conjugates comprises a plurality of conjugates with binding specificity for the same biomarker;
wherein each population of binding moiety-oligonucleotide conjugates comprises a plurality of conjugates comprising an identifier nucleotide sequence which is indicative of the biomarker to which the binding moiety has binding specificity; and
wherein each binding moiety in a population is conjugated to the same number of oligonucleotide moieties;
optionally wherein each conjugate in a population comprises the same binding moiety; and/or
wherein the system comprises two or more populations of binding moiety-oligonucleotide conjugates, wherein the two or more populations of conjugates have binding specificity for different biomarkers.

95. The system of claim 94 comprising populations of binding moiety-oligonucleotide conjugates with specificity to biomarkers in a biomarker signature of a disease state.

96. The system of claim 94 further comprising one or more components from the group consisting of:
one or more substrate(s);
means for immobilising biomarkers to the substrate;
a means for detecting and/or quantifying the identifier nucleotide sequences within the oligonucleotide moieties of the binding moiety-oligonucleotide conjugates;
superparamagnetic polymer particles;
means for biotinylating biomarkers and/or means for coating the substrate with streptavidin or avidin;
PCR primers for amplifying the identifier nucleotide sequences within the oligonucleotide moieties of the binding moiety-oligonucleotide conjugates; and
software or an algorithm for analysing nucleotide sequence data and categorising the biological sample.

97. A kit of parts for manufacturing a binding moiety-oligonucleotide conjugate, wherein the kit of parts comprises:
(a) one or more binding moieties as defined in claim 90; and/or
(b) one or more oligonucleotide moieties as defined in claim 90, and
optionally further comprises a polypeptide having sortase activity.

98. A method of diagnosis and/or prognosis of a disease state in a subject, comprising the steps:
(a) providing a biological sample from the subject to be tested; and
(b) detecting and/or quantifying one or more biomarkers(s) of interest in the sample using the method according to claim 79,
wherein the presence and/or quantity of the one or more biomarkers(s) of interest is indicative of the disease state,
optionally, wherein the disease state is selected from the group consisting of:
(a) the presence or absence of a disease;
(b) the stage or extent of a disease progression and/or level of disease activity; and
(c) the responsiveness to a therapeutic agent for treating a disease; and/or
wherein the disease is selected from the group consisting of a cancer; an autoimmune disease; a blood disease; an infectious disease; and a genetic disease;
optionally further comprising the step of administering to the subject an effective treatment for the disease, following the diagnosis or prognosis of the disease state.
